(19) United States
(12) Patent Application Publication
(10) Pub. No.: US 2018/0150597 A1
(43) Pub. Date: May 31, 2018

(54) METHOD FOR OPTIMAL DESIGN OF POLYNUCLEOTIDES SEQUENCES FOR ANALYSIS OF SPECIFIC EVENTS IN ANY GENETIC REGION OF INTEREST

(71) Applicant: Genomic Vision SA, Bagneux (FR)

(72) Inventors: Sara Berthoumieux, Paris (FR); Yannick Fourne, Paris (FR); Jun Komatsu, Bagneux (FR); Frederic Fer, Paris (FR); Aaron Bensimon, Antony (FR)

(73) Assignee: Genomic Vision SA, Bagneux (FR)

(21) Appl. No.: 15/826,035

(22) Filed: Nov. 29, 2017

Related U.S. Application Data
(60) Provisional application No. 62/427,580, filed on Nov. 29, 2016.

(51) Int. Cl. G06F 19/20; G06F 19/22; G06F 19/18; C12Q 1/68

(52) U.S. Cl. CPC G06F 19/20; C12Q 1/6874; G06F 19/18; G06F 19/22

(57) ABSTRACT

Methods including in-silico steps for design and synthesis of Genomic Morse Code (“GMC”) probes including design of combinations of polynucleotide sequences and labelling colors for analysis of large rearrangements in targeted genetic regions as well as allele characterization of complex regions and localization of events such as replication, DNA reparation or epigenetics in particular regions. Color-encoded sets of probes that produce characteristic or unique color patterns when painted on a target nucleic acid sequence. Methods for using color-encoded sets of probes.

### BACKGROUND OF THE INVENTION

### Field of the Invention

Methods for analysis of specific events in a genetic region of interest, including genetic rearrangements, characterization of alleles especially in complex regions, and localization of events such as DNA replication, DNA reparation or DNA epigenetics. Engineered polynucleotides designed in silico which may be labelled with different colors or markers useful for analysis of these genetic events.

### Description of the Related Art

Genomic biomarker research often involves the study of replication or identification of genetic structural variations in regions with complex repetitions; phenomena that are poorly detected with standard sequencing technologies. Single-molecule technologies such as Molecular Combing, optical mapping and FISH can overcome these difficulties; see Michalet et al., 1997; Jing et al., 1998; Gal and Pardue, 1969; Bauman et al., 1980.

In particular, Molecular Combing allows direct visualization of targeted regions of interest with a unique detection strategy, the Genomic Morse Code (“GMC”); see U.S. Pat. No. 7,985,542 B2, U.S. Pat. No. 9,133,514 B2, each incorporated by reference. The fluorescent GMC provides a specific coding pattern that combines both color and probe length for the direct visualization of loci of interest. GMC patterns can be designed specifically for any genetic region or any set of multiple genetic regions of interest and are adaptable to the exact nature of the scientific hypothesis investigated. Such an approach using a pattern of colored probes could be applied to FISH technology as well. Although optical mapping technology is not currently capable of working with a specifically designed set of probes, recent results obtained with coupling of CRISPR-CAS9 and nick-labeling approaches renders probe pattern design possible in the near future; McCaffrey et al., 2015. However, until now, no methodology has been available to effectively, efficiently or economically identify the color-pattern design of a combination of polynucleotide sequences required for any of these technologies.

Properly designed probe patterns can be used for detection of genetic rearrangements, for companion diagnostic products or localization of replication kinetic events onto specific genetic regions. For example, the GMC approach with molecular combing technology enabled the identification of large rearrangements in BRCA1 and BRCA2 regions; see Gad et al., 2001; Cheeseman et al., 2012; Puget et al., 2002; and the correlation study between replication kinetics and replication origin positions; see Lebofsky et al., 2006. Lebofsky shows an example of GMC with mono-color probes with a particular combination distances between probes that enable the localization of replication signals. However, no methodology was described for the design of the required GMC.

The constraints encountered when designing probe patterns are twofold: regulation of repeat sequence detection and fragmentation of target polynucleotides.

The first one is the presence of abundant amounts of repeat sequences in polynucleotide, especially in genomic DNA. Since a DNA sequence is composed of only 4 different bases, very short stretches of sequence, such as restriction enzyme site (4-8 bases), appear with certain density all over genomic sequence. Although the distribution pattern of such short sequence generates naturally identifiable local sub-patterns, which are sometimes employed by other optical mapping assays, it obliges one to analyze massive numbers of sub-patterns in the entire genome in order to get sufficient information from the loci of interest.

In order to obtain information efficiently from the region of interest (“ROI”), a polynucleotide sequence or set of polynucleotide sequences can be selected from a locus of interest for a target of labelling. As a genomic DNA sequence, especially a higher eukaryote genomic DNA sequence, is not random at all, simple increase of size of polynucleotide sequence does not necessarily guarantee a uniqueness of the polynucleotide sequence in given genome sequence. Both short and long interspersed nuclear elements (SINE and LINE) are stretches of DNA sequences usually having several hundred to thousand bases which are highly repeated and which appear all over the genome.

Inclusion of such sequences in the set of polynucleotide sequences defining probe pattern must be regulated. This can be done by exclusion of high copy repeats either when one probe polynucleotide is synthesized; see Swennenhuis, 2012; or when polynucleotide sequences are designed; see Beliveau, 2012; Bienko, 2013. Segmental duplications, such as low copy repeats, that can be several hundred kilo bases or more, cause duplication of all or parts of probe signals if the locus of interest is involved in the duplication. In that case, the design of the probe pattern must either exclude polynucleotide sequences that are part of segmental duplications or generate patterns that enable the discrimination between data from region of interest and data from duplicated loci.

The second constraint is the fragmentation of testing polynucleotides, such as the genomic DNA of cell lines or individuals, during sample preparation. In case of scientific interest over a set of multiple genetic regions, each region probe pattern must be unique and identifiable from patterns of other regions. When the length of each region of interest (“ROI”) is much smaller than the representative size of prepared testing polynucleotide, the experimentally obtained signals of set of polynucleotide sequence probes are expected to contain the complete probe pattern of each ROI. It is then possible to detect the occurrence of a genomic rearrangement when the signal pattern is not identical to the theoretical probe pattern. However, when the size of the ROI is close to or bigger than the representative size of prepared sample polynucleotide, significant amount of signals will only contain subparts of theoretical pattern due to physical fragmentation of genomic DNA during sample preparation process. This fragmentation of a genomic DNA sample can force reconstruction of the whole information of ROI from partial local information. This means that not only ROI complete probe patterns but also subparts of each ROI probe pattern have to be unique and distinguishable from any other subparts of ROI patterns. There are many works and algorithms for assembly of partial information for reconstruction of DNA sequence from sequencing or optical mapping signals; see Flicek and Birney, 2009; Hastie et al., 2013. However, the problematic of a method for design of probe pattern that optimizes efficiency of self-reconstruction from partial information has been hardly studied.

Although the distribution of repeated elements on the regions of interest has been previously reported as important information for the design of probe patterns, the existing patents about Genomic Morse Code and other types of probe combinations; see Lebofsky, 2007; Komatsu, 2016; do not consider the analysis of segmental duplications outside the region(s) or the constraint of subpattern uniqueness along the ROI(s) in the design process.

### BRIEF SUMMARY OF THE INVENTION

The invention is directed to methods for designing and using coded multi-labelled color probes as based on the Genomic Morse Code approach as well as the designed or engineered probes themselves. The invention is also directed to a method for analysis of specific events in a genetic region of interest and polynucleotides designed therefore. One prominent embodiment is a method for designing color-coded Genetic Morse Code (“GMC”) probe(s) comprising identifying a sequence of a nucleic acid target region of interest in a genomic, chromosomal or other nucleic acid sample, subdividing the sequence of the target region of interest by defining a set of subsequences, identifying duplicate subsequences in the set of defined subsequences inside the target region of interest, designing the minimal set of GMC probe(s) that bind to the full nucleic acid target region of interest, wherein said designed GMC probe(s) produce a unique or characteristic color pattern when bound to the nucleic acid target region of interest; and, optionally, synthesizing said designed GMC probe(s). Synthesized GMC probe(s) may be contacted with a polynucleotide sequence under conditions suitable for their binding and identification of a target region of interest, for example, they may be employed in a Molecular Combing procedure of genomic DNA.

The method also comprises identifying duplicate subsequences outside the target region of interest and designing GMC probe(s) that bind to the nucleic acid target region of interest but that do not bind to these duplicate subsequences or that identify them with one or more specific colors. The composition of successive GMC probe(s) provides a unique signature for detection of the presence, absence or modification of targeted regions. Moreover, subparts of this sequence of successive colored elements are also uniquely defined and enable the exact localization of partial or complete color-coded compositions. The present invention concerns the definition of the technical steps allowing the obtaining of ultra-specific composition of successive colored reagents useful for detection of presence, absence or modification of targeted regions in the genome using molecular combing and hybridization techniques.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Overall scheme of design tool for color-coded GMCs providing selective or unique probe patterns.

FIG. 2: Scheme of algorithm that identifies problematic segmental duplications. “ROI” stands for “region of interest”. One or more of these steps is or may be performed on a computer.

FIG. 3: Scheme for algorithmic post-processing of genome alignment results. One or more of these steps is or may be performed on a computer.

FIG. 4: Scheme for algorithmic step of identification of problematic sequences. One or more of these steps is or may be performed on a computer.

FIG. 5: Scheme of algorithm that defines color-coded probe patterns. One or more of these steps is or may be performed on a computer.

FIG. 6: Relative positions of DNA probes to hybridize along the region of interest. Mb stands for megabases. Each probe pattern is monocolor. The colors of the probes are graphical representations and do not reflect real colors obtained on experimental results.

FIGS. 7A and 7B: Probe patterns covering 2 genes involved in HNPCC, designed from the method described in the patent about probe combinations for detection of large rearrangement; Komatsu, 2007. Relative positions of DNA probes are according to GRCh19/hg19 human genome. The upper probe pattern covers MLH1 gene while the second one covers PMS2 gene. Graphical representations of probe patterns were obtained using the Genome Browser webtool; see Genome Browser (2017). FIGS. 7A and 7B are overlapping panels.

FIG. 8: Example of experimental signal which localization on probe patterns cannot be determined. The signal of 40 kb could either be a sub part of the PMS2 probe pattern (situated above experimental signal) or a sub part of MLH1 probe pattern (situated below the experimental signal). Graphical representations of probe patterns were obtained using the Genome Browser webtool; see Genome Browser (2017).

FIG. 9: Segmental duplication of the about first 36 kb of the GMC covering PMS2 gene. Graphical representations of probe patterns were obtained using the Genome Browser webtool; incorporated by reference to Genome Browser (2017).

FIGS. 10A and 10B: Probe patterns of 2 regions of interest, each covering a gene involved in HNPCC (MLH1 for the upper one, PMS2 for the lower). The probe patterns are designed using the probe pattern method presented in this document. Relative positions of DNA probes are according to GRCh19/hg19 human genome. Graphical representations of probe patterns were obtained using the Genome Browser webtool; see Genome Browser (2017). FIGS. 10A and 10B are overlapping panels.

FIG. 11A: Probe pattern covering SMA region. Relative positions of DNA probes are according to GRCh38/hg38 human genome (Rosenbloom et al., 2015). The relative positions of genes localized on the SMA locus are indicated below the probe pattern. Graphical representation of the probe pattern was obtained using the Genome Browser webtool; see Genome Browser (2017).

FIG. 11B: Example of experimental signals obtained by molecular combing and hybridization of the probes shown in FIG. 11A. The signals are manually aligned with each other in order to reconstitute the probe pattern of the SMA locus.

FIG. 12: Computer system upon which embodiments of the present disclosure may be implemented.

FIG. 13A: Probe pattern of target region coverage (above probe pattern) as well as probe pattern synthesized (below probe pattern) on target region. Relative positions of DNA probes along the region of interest are specified. Kb stands for kilobases. The relative positions of genes and pseudo-genes are localized on the target locus and indicated below the probe pattern. In the figure, “GENE” stands for the gene of interest and “PSGE1”, “PSGE2”, ‘PSGE3”, “PSGE4” and “PSGE5” for the 5 pseudo-genes of gene “GENE”. Graphical representations of the probe pattern were obtained using the Genome Browser webtool; see Genome Browser (2017).

FIG. 13B: Example of experimental signals obtained by molecular combing and hybridization of the probes shown in FIG. 13A.

### DETAILED DESCRIPTION OF THE INVENTION

The inventors disclose herein an in-silico tool that designs a set of sequences or biomarkers that is advantageous or even optimal for the detection of specific events (known, newly identified, or unknown structural variations, characterization of a complex region, replication signal localization, etc. . . . ) in any (set of) genetic region(s) of interest above 0.5-1 kb each and for any biomolecular technology.

In the context of application to molecular combing technology, the tool provides probe patterns based on a sequence of probes of different colors and lengths. The resultant probe patterns provide efficient visualization and unambiguous localization of signals obtained by molecular combing and fluorescent hybridization of the designed probes. The probes selected by this method can be used as biomarkers for the identification and the localization of such sequences on a gene or a region corresponding to several genes. The visual interaction between a biomarker obtained by this method and a DNA fragment to be tested can be shown on linearized or stretched polynucleotidic molecules.

Genomic biomarker research can involve the study of replication or identification of genetic structural variations; phenomena that are poorly detected with standard sequencing technologies in regions with complex repetitions.

Single-molecule technologies for polynucleotide sequence analysis can overcome these difficulties. In particular, Molecular Combing allows direct visualization of targeted regions of interest with a unique detection strategy, the Genomic Morse Code (“GMC”). The fluorescent GMC provides a specific coding pattern that combines both color and probe length for the direct visualization of a locus or loci of interest. In the context of Molecular Combing, GMC patterns can be designed specifically for any genetic region of interest and are adaptable to the exact nature of the scientific hypothesis investigated.

The constraints encountered when designing GMCs are twofold. Firstly, hybridization feasibility depends on the genetic complexity of loci of interest and more particularly the presence of repeat elements and segmental duplications. Secondly, DNA breakage during the extraction step can render localization of partial signals problematic. Consequently, the inventors provide an in-silico GMC design tool for characterization of specific loci of interest. In addition, the tool can design GMC used for localization of events such as replication, DNA reparation or epigenetics.

This tool tackles both technical issues by linking bioinformatics and combinatorial in silico analysis. First, a bioinformatics algorithm excludes sequences rich in repeated elements from design. Segmental duplications are identified and taken into account during GMC design without being systematically excluded from the region of interest. Moreover, if required, duplicated sequences outside the target genomic region can also be specifically labelled during the GMC design process in order to differentiate them during downstream analysis. Second, the algorithm comprises a combinatorial element that designs a color-coded GMC with a unique color pattern. The unique color coding allows a non-ambiguous localization of signals from the loci of interest, whether or not the GMC is fragmented by DNA breakage during extraction. The composition of successive colored reagents provides a unique signature for detection of the presence, absence or modification of targeted regions. Moreover, subparts of this sequence of successive colored elements are also uniquely defined and enable the exact localization of partial or complete color-coded compositions.

This algorithm provides a combination of polynucleotide sequences, distinguishable by their color and/or length patterns, for biomarker analysis or the detection of specific events (such as known or unknown structural variations, replication signal localization, etc. . . . ) with any biomolecular technology. Efficient visualization and unambiguous signal localization of the resultant sequence combinations are guaranteed. The present invention concerns the definition of the technical steps allowing the obtaining of ultra-specific composition of successive colored reagents useful for detection of presence, absence or modification of targeted regions in the genome using molecular combing and hybridization techniques.

The terms “genome” or “genomic” as used herein are simplifications. It should be understood that the methods such as Molecular Combing, described herein may be practiced with other DNA or nucleic acid sequences capable of being attached to a combing surface including engineered nucleic acids, artificial chromosomes, etc. The term “duplicate” or “duplicated” or “repeat” or “repeated” is intended to indicate more than one instance, for example, 2, 3, 4, 5, 6, 7, 8, 9, 10 or more instances of a particular sequence. These terms denote the presence of repeated and duplicated sequences and are not to be construed as limiting such sequences to those made by any particular biological mechanism.

Genomic Morse Code or GMC is a general tool and method for comprehensive analysis and physical mapping of one or more target regions on a nucleic acid, such as a target region of a stretched nucleic acid, such as a DNA molecule stretched using molecular combing. GMC probes generally comprise a combination of fluorescent probes of different colors and sizes, designed to recognize a selected region of interest. As a result, the DNA sequence to be analyzed is labelled with the combination of “dashes and dots”, creating a “Morse Code” specific to a target gene and its flanking regions. However, as explained herein, the utility of a set of GMC probes may be compromised when target nucleic acid contains duplicated or repeated sequences or when target DNA is broken.

Genomic Morse Code provides a comprehensive analysis and physical mapping of target regions on stretched DNA. Combed DNA is hybridized with a combination of fluorescent probes of different colors and sizes, designed to recognize a selected region of interest. As a result, the DNA sequence to be analyzed is labelled with the combination of “dashes and dots”, creating a “Morse Code” specific to a target gene and its flanking regions. The strategy underlying GMC is to use the spatial distribution of the probes to provide additional information than simply measuring just the probes. The recognition of different motifs in the Genomic Morse Code (e.g., probe pattern painted on a target nucleic acid) is not only based on probe size and color, but also on their order and the distances between them. The identical stretching of the DNA allows for accurate and reproducible measurements of the length of the probes as well as the gaps separating them. Any change in the observed pattern compared to the Genomic Morse Code of a reference indicates the presence of a rearrangement in the target locus. Amplifications, deletions, repeats, inversions and translocations can be identified and analyzed depending on the chosen Genetic Morse Code design with no bias due to sequence content. The GMC method allows the detection of balanced rearrangements often missed by other methods and also provides information about the location and the exact number of copies found. GMC probes are defined as polynucleotidic sequences which are labelled according to the GMC method. The present invention provides GMC probes having superior properties to those described previously, such as having superior specificity for loci of interest compared to conventional GMC probes.

Known methods for designing and making GMC probes and molecular combing procedures are described by US 2016/0047006, US 2016/0040249, US 2016/0040220, US 2015/0197816, US 2014/0220160, US 2013/0130246, and US 2012/0076871, US 2011/0287423, US 2010/0041036 (now U.S. Pat. No. 8,586,723) and US 2008/0064114 (now U.S. Pat. No. 7,985,542) each of which is incorporated by reference.

The term Genomic Morse Code may be used in conjunction with the set of probes that when bound to a target locus or loci produce a particular pattern of colors or particular detectable labelling pattern or, alternatively, to identify the color or detectable label pattern exhibited by a target nucleic acid contacted with these probes. This term also encompasses the definitions of Genetic Morse Codes used in U.S. Pat. No. 8,586,723 (issued 2013) and U.S. Pat. No. 7,985,542 (issued 2011). In one embodiment of these cited patents, GMC probes comprise at least three different probes each distanced from one another by either a small gap of 25-30 kb or by a long gap between 55-70 kb and having an assigned color or label. Other numbers and combinations of probes may be used with different spacings, such as a combination of two, three, four, five, six, seven, eight, nine, ten or more probes that may exhibit a characteristic or unique color pattern when painted on a target nucleic acid such as genomic or chromosomal DNA. GMC probes can also be consecutive and have no spacing between them, or be separated from gaps which sizes range from 1 to hundreds of kilobases. Probe sizes can also vary from 500 base pairs to hundreds of kilobases. For example, probe sizes can be comprised between 100 kilobases and 800 kilobases, for example, a probe may be 100, 200, 300, 400, 500, 600, 700, or 800 kb.

Methods of GMC Probe Design.

Some methods for design of GMC probes that do not include one or more of the design steps of the invention include:

A method of detection of the presence of at least one domain of interest on a macromolecule to test, comprising: a) determining beforehand at least two target regions on the domain of interest, designing and obtaining corresponding labeled probes of each target region, named set of probe of the domain of interest, the position of these probes one compared to the others being chosen and forming the specific signature of said domain of interest on the macromolecule to test; b) after spreading of the macromolecule to test on which the probes obtained in step a) are bound, detection of the position one compared to the others of the probes bound on the linearized macromolecule, the detection of the signature of a domain of interest indicating the presence of said domain of interest on the macromolecule to test, and conversely the absence of detection of signature or part of signature of a domain of interest indicating the absence of said domain or part of said domain of interest on the macromolecule to test.

A method of detection of the presence of at least one domain of interest on a macromolecule to test, comprising: a) determining beforehand at least two target regions on the domain of interest, designing and obtaining corresponding labeled probes of each target region, named set of probe of the domain of interest, the position of these probes one compared to the others being chosen and forming the specific signature of said domain of interest on the macromolecule to test; b) after spreading of the macromolecule to test on which the probes obtained in step a) are bound, detection of the position one compared to the others of the probes bound on the linearized macromolecule, the detection of the signature of a domain of interest indicating the presence of said domain of interest on the macromolecule to test, and conversely the absence of detection of signature or part of signature of a domain of interest indicating the absence of said domain or part of said domain of interest on the macromolecule to test.

Color-Coding/Probe Labels.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radishperoxidase, alkalinephosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, hereby incorporated by reference. One skilled in the art may replace color-coded labels with other detectable labels disclosed herein.

A fluorescent label is preferred because it provides a very strong signal with low background. It is also optically detectable at high resolution and sensitivity through a quick scanning procedure.

The probe can all be labeled with a single label, e.g., a single fluorescent label. Alternatively, in another embodiment, different probes can be simultaneously hybridized where each probe has a different label. For instance, one target could have a green fluorescent label and a second target could have a red fluorescent label. The scanning step will distinguish sites of binding of the red label from those binding the green fluorescent label. Each probe (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

A wide variety of suitable dyes are available, being primarily chosen to provide an intense color with minimal absorption by their surroundings. Illustrative dye types include quinoline dyes, triarylmethane dyes, acridine dyes, alizarine dyes, phthaleins, insect dyes, azo dyes, anthraquinoid dyes, cyanine dyes, phenazathionium dyes, and phenazoxonium dyes.

A wide variety of fluorescers can be employed either alone or, alternatively, in conjunction with quencher molecules. Fluorescers of interest fall into a variety of categories having certain primary functionalities. These primary functionalities include 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopyridinium salts, hellebrigenin, tetracycline, sterophenol, benzimidzaolylphenylamine, 2-oxo-3-chromen, indole, xanthen, 7-hydroxycoumarin, phenoxazine, salicylate, strophanthidin, porphyrins, triarylmethanes and flavin.

Individual fluorescent compounds which have functionalities for linking or which can be modified to incorporate such functionalities include, e.g., dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene: 4-acetamido-4-isothiocyanato-stilbene-2,2′-disulfonic acid; pyrene-3-sulfonic acid; 2-toluidinonaphthalene-6-sulfonate; N-phenyl, N-methyl 2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4(3′pyrenyl)butyrate; d-3-aminodesoxy-equilenin; 12-(9′anthroyl)stearate; 2-methylanthracene; 9-vinylanthracene; 2,2′(vinylene-p-phenylene)bisbenzoxazole; p-bis[2-(4-methyl-5-phenyl-oxazolyl)]benzene; 6-dimethyl amino-1,2-benzophenazin; retinol; bis(3′-aminopyridinium) 1,10-decandiyl diiodide; sulfonaphthylhydrazone of hellibrienin; chlorotetracycline; N(7-dimethylamino-4-methyl-2-oxo-3-chromenyl)maleimide; N-[p-(2-benzimidazolyl)-phenyl]maleimide; N-(4-fluoranthyl)maleimide; bis(homovanillic acid); resazarin; 4-chloro-7-nitro-2,1,3benzooxadiazole; merocyanine 540; resorufin; rose bengal; and 2,4-diphenyl-3(2H)-furanone.

In particular fluorescent labels according to the present invention are 1-Chloro-9,10-bis(phenylethynyl)anthracene, 5,12-Bis(phenylethynyl)naphthacene, 9,10-Bis(phenylethynyl)anthracene, Acridine orange, Auramine O, Benzanthrone, Coumarin, 4′,6-Diamidino-2-phenylindole (DAPI), Ethidium bromide, Fluorescein, Green fluorescent protein, Hoechst stain, Indian Yellow, Luciferin, Phycobilin, Phycoerythrin, Rhodamine, Rubrene, Stilbene, TSQ, Texas Red, and Umbelliferone.

Desirably, fluorescers should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single label can provide for a plurality of measurable events.

According to the present invention, when the labelling is made with fluorescent label, the reading of signals is made by fluorescent detection the fluorescently labelled probe is excited by light and the emission of the excitation is then detected by a photosensor such as CCD camera equipped which appropriate emission filters which captures a digital image and allows further data analysis.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. A diverse number of families of compounds have been found to provide chemiluminescence under a variety of conditions. One family of compounds is 2,3-dihydro-1,-4-phthalazinedione. The most popular compound is luminol, which is the 5-amino compound. Other members of the family include the 5-amino-6,7,8-trimethoxy- and the dimethylamino[ca]benz analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence can also be obtained with oxalates, usually oxalyl active esters, e.g., p-nitrophenyl and a peroxide, e.g., hydrogen peroxide, under basic conditions. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label may be added to the probe (or target, which is in particular nucleic acid(s)) prior to, or after the hybridization. So called “direct labels” are detectable labels that are directly attached to or incorporated into the probe prior to hybridization. In contrast, so called “indirect labels” are joined to the hybrid duplex after hybridization. Often, the indirect label is attached to a binding moiety that has been attached to the probe prior to the hybridization. Thus, for example, the probe may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993), incorporated by reference.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

Concerning end-labeling probes, in many applications it is useful to directly label probes without having to go through amplification, transcription or other conversion step. In general, end-labeling methods permit the optimization of the size of the nucleic acid to be labeled. End-labeling methods also decrease the sequence bias sometimes associated with polymerase-facilitated labeling methods. End labeling can be performed using terminal transferase (TdT).

End labeling can also be accomplished by ligating a labeled oligonucleotide or analog thereof to the end of a probe. Other end-labeling methods include the creation of a labeled or unlabeled “tail” for the nucleic acid using ligase or terminal transferase, for example. The tailed nucleic acid is then exposed to a labeled moiety that will preferentially associate with the tail. The tail and the moiety that preferentially associates with the tail can be a polymer such as a nucleic acid, peptide, or carbohydrate. The tail and its recognition moiety can be anything that permits recognition between the two, and includes molecules having ligand-substrate relationships such as haptens, epitopes, antibodies, enzymes and their substrates, and complementary nucleic acids and analogs thereof.

The labels associated with the tail or the tail recognition moiety includes detectable moieties. When the tail and its recognition moiety are both labelled, the respective labels associated with each can themselves have a ligand-substrate relationship. The respective labels can also comprise energy transfer reagents such as dyes having different spectroscopic characteristics. The energy transfer pair can be chosen to obtain the desired combined spectral characteristics. For example, a first dye that absorbs at a wavelength shorter than that absorbed by the second dye can, upon absorption at that shorter wavelength, transfer energy to the second dye. The second dye then emits electromagnetic radiation at a wavelength longer than would have been emitted by the first dye alone. Energy transfer reagents can be particularly useful in two-color labeling schemes such as those set forth in a copending U.S. patent application, filed Dec. 23, 1996, and which is a continuation-in-part of U.S. Ser. No. 08/529,115, filed Sep. 15, 1995, and International Appln. No. WO 96/14839, filed Sep. 13, 1996, which is also a continuation-in-part of U.S. Ser. No. 08/670,118, filed on Jun. 25, 1996, which is a division of U.S. Ser. No. 08/168,904, filed Dec. 15, 1993, which is a continuation of U.S. Ser. No. 07/624,114, filed Dec. 6, 1990. U.S. Ser. No. 07/624,114 is a CIP of U.S. Ser. No. 07/362,901, filed Jun. 7, 1990, incorporated herein by reference.

In one embodiment of these cited patents, when the labeling is made with radioactive label, the reading of signals is made by radioactive detection. Radioactive detection can be made with X-ray film or a phosphorimager. Examples of radioactive labels according to the present invention are ³H, ¹²⁵I, ³⁵S, ¹⁴C, or ³²P.

In a preferred embodiment of the cited patents, the probes are labeled with one or more fluorescent labels. In another preferred embodiment of the cited patents, the probes are labeled with radioactive label(s).

According to the present invention, in the case of the probes are labeled with at least two different labels the signature of a domain of interest results of the succession of labels.

The color-coded GMC probe(s) of the invention may be used to diagnose viral infections by detection of genomic or infectious viral DNA by molecular combing, for the detection of amplified sequences, such as sequence amplification in BRCA loci, for the detection of breakpoints in rearranged genomic DNA, for detection, visualization and mapping of genomic rearrangements, for example in breast or ovarian cancer genes or BRCA1 or BRCA2 loci, for detection, quantification, and mapping damaged DNA or repaired DNA.

Target Nucleic Acid Lengths, Probe Lengths and Spacings.

There is no upper limitation on the length of target DNA regions to be investigated using the GMC probe(s) of the invention other than the maximal length of chromosomal or other nucleic acids of interest. Regions of at least 1, 10, 20, 30, 40, 50, 60, 70, 80, 90, 100, 500, 750, 1,000, 2,000 kb in length may be investigated. Consequently, there is no maximal length for GMC probe(s). In the case of Molecular Combing methods, detection resolution may require probes at least 500 kb in length, for example, 3 kb or 160 kb as shown in the Examples. Gaps between GMC probes in a set of probes providing a characteristic or unique probe pattern can range from 0 kb (e.g., for SMA, MLH1 or PSM2 regions), to 200 kb for a replication probe pattern or set of GMCs. Longer gaps of least 1, 10, 20, 30, 40, 50, 60, 70, 80, 90, 100, 500, 750, 1,000, or more 2,000 kb are also contemplated.

Kits Containing GMC Probe(s).

A kit for the detection of at least one domain or locus of interest of a nucleic acid such as genomic DNA will contain the color-coded GMC probe(s) according to the invention. Other ingredients may include equipment and reagents for sample preparation including DNA extraction equipment that provides purified, very high molecular weight DNA (e.g., median size of 100 kb) suitable for Molecular Combing; equipment and reagents for Molecular Combing, such as a vinyl silane treated glass surface (e.g., a coverslip) and equipment or a system for stretching DNA; equipment and devices (e.g., a scanner) for reading target DNA contacted with GMC probe(s) and software or computer equipment for analyzing, processing and storing these data. Kits may also include instructions for use or marketing or promotional materials.

Hybridization.

As used herein, the term “hybridization”, “hybridizes to” or “hybridizing” is intended to describe conditions for moderate stringency or high stringency hybridization, preferably where the hybridization and washing conditions permit nucleotide sequences at least 60% homologous to each other to remain hybridized to each other.

Preferably, the conditions are such that sequences at least about 70%, more preferably at least about 80%, even more preferably at least about 85%, 90%, 95% or 98% homologous to each other typically remain hybridized to each other. Stringent conditions are known to those skilled in the art and can be found in Current Protocols in Molecular Biology, John Wiley & Sons, N.Y. (1989), 6.3.1-6.3.6.

By nucleic sequences having a percentage of identity of at least 80%, preferably 85%, 90%, 95% and 98%, after optimum alignment with a preferred sequence, it is intended to indicate the nucleic sequences having, with respect to the reference nucleic sequence, certain modifications such as, in particular, a deletion, a truncation, an elongation, a chimeric fusion and/or a substitution, especially point substitution. It preferably concerns sequences in which the sequences code for the same amino acid sequences as the reference sequence, this being connected to the degeneracy of the genetic code, or complementary sequences which are capable of hybridizing specifically with the reference sequences, preferably under conditions of high stringency, especially such as defined below.

Hybridization under conditions of high stringency signifies that the temperature conditions and ionic strength conditions are chosen in such a way that they allow the maintenance of the hybridization between two fragments of complementary DNA. By way of illustration, conditions of high stringency of the hybridization step for the purposes of defining the polynucleotide fragments described above are advantageously the following.

The DNA-DNA or DNA-RNA hybridization is carried out in two steps: (1) prehybridization at 42° C. for 3 hours in phosphate buffer (20 mM, pH 7.5) containing 5.times. SSC (1×SSC corresponds to a 0.15 M NaCl+0.015 M sodium citrate solution), 50% of formamide, 7% of sodium dodecyl sulfate (SDS), 10×Denhardt's, 5% of dextran sulfate and 1% of salmon sperm DNA; (2) actual hybridization for 20 hours at a temperature dependent on the size of the probe (i.e.: 42° C., for a probe size>100 nucleotides) followed by 2 washes of 20 minutes at 20° C. in 2.times. SSC+2% of SDS, 1 wash of 20 minutes at 20° C. in 0.1.times. SSC+0.1% of SDS. The last wash is carried out in 0.1.times. SSC+0.1% of SDS for 30 minutes at 60° C. for a probe size >100 nucleotides. The hybridization conditions of high stringency described above for a polynucleotide of defined size can be adapted by the person skilled in the art for oligonucleotides of greater or smaller size, according to the teaching of Sambrook et al., (1989, Molecular cloning: a laboratory manual. 2nd Ed. Cold Spring Harbor). In an embodiment, the probes are oligonucleotides of at least 15 nucleotides, preferably at least 1 kb more preferably between 1 to 10 kb, even more preferably between 4 to 10 kb. Since maximal resolution on combed DNA is 1-4 kb, probes according to present invention are preferably of at least 4 kb. In some embodiments, linearization of the macromolecule is made before or after binding of the probes on the macromolecules; in others the linearization of the macromolecule is made by molecular combing or Fiber Fish.

Genetic Diseases and Disorders.

Nucleic acids associated with genetic diseases and disorders may be detected using the GMC probe(s) of the invention, for example, in combination with Molecular Combing of genomic DNA. Genetic diseases or disorders that may be detected, characterized, or quantified using the GMC probe(s) and methods of the invention include, but are not limited to Achondroplasia, Alpha-1 Antitrypsin Deficiency, Antiphospholipid Syndrome, Autism, Autosomal Dominant Polycystic Kidney Disease, Breast cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Facio-Scapulo-Humeral Dystrophy (FSHD), Familial Mediterranean Fever, Fragile X Syndrome, Gaucher Disease, Hemochromatosis, Hemophilia, Holoprosencephaly, Huntington's disease, Klinefelter syndrome, Leber Congenital Amaurosis, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Tay-Sachs, Thalassemia, Trimethylaminuria, Turner Syndrome, Velocardiofacial Syndrome, WAGR Syndrome, and Wilson Disease.

The GMC probe(s) (e.g., set of probes producing a characteristic or unique pattern when painted on to a target nucleic acid) and methods of the invention may be employed to detect, characterize, assess or quantify genome or gene editing events in a polynucleotide, genome, exon, intron, or gene of choice. Specific kinds of genes include, but are not limited to prokaryotic or eukaryotic genes or genomes, yeast or fungal genomes or genes, plant or algae genes, invertebrate or vertebrate genes, genes from fish, amphibians, reptiles, birds including chickens, turkeys and ducks, mammalian genes including those of domesticated animals, such as horses, cattle, cows, goats, sheep, llamas, camels, or pigs. Such genes include any of the following a mammalian β globin gene (HBB), a gamma globin gene (HBG1), a B-cell lymphoma/leukemia 11A (BCL11A) gene, a Kruppel-like factor 1 (KLF1) gene, a CCR5 gene, a CXCR4 gene, a PPP1R12C (AAVS1) gene, an hypoxanthine phosphoribosyltransferase (HPRT) gene, an albumin gene, a Factor VIII gene, a Factor IX gene, a Leucine-rich repeat kinase 2 (LRRK2) gene, a Huntingtin (Htt) gene, a rhodopsin (RHO) gene, a Cystic Fibrosis Transmembrane Conductance Regulator (CFTR) gene, a surfactant protein B gene (SFTPB), a T-cell receptor alpha (TRAC) gene, a T-cell receptor beta (TRBC) gene, a programmed cell death 1 (PD1) gene, a Cytotoxic T-Lymphocyte Antigen 4 (CTLA-4) gene, an human leukocyte antigen (HLA) A gene, an HLA B gene, an HLA C gene, an HLA-DPA gene, an HLA-DQ gene, an HLA-DRA gene, a LMP7 gene, a Transporter associated with Antigen Processing (TAP) 1 gene, a TAP2 gene, a tapasin gene (TAPBP), a class II major histocompatibility complex transactivator (CIITA) gene, a dystrophin gene (DMD), a glucocorticoid receptor gene (GR), an IL2RG gene, a centrosomal protein of 290 kDa (CEP290), Double homeobox 4 (DUX4) and an RFX5 gene. Such genes also include a plant FAD2 gene, a plant FAD3 gene, a plant ZP15 gene, a plant KASII gene, a plant MDH gene, and a plant EPSPS gene.

Molecular Combing technology has been disclosed in various patents and scientific publications, for example in U.S. Pat. No. 6,303,296, WO 9818959, WO 0073503, U.S. 2006/257910, U.S.2004/033510, U.S. Pat. No. 6,130,044, U.S. Pat. No. 6,225,055, U.S. Pat. No. 6,054,327, WO 2008/028931, WO 2010/035140, and in (Michalet, Ekong et al. 1997; Herrick, Michalet et al. 2000; Herrick, Stanislawski et al. 2000; Gad, Aurias et al. 2001; Gad, Caux-Moncoutier et al. 2002; Gad, Klinger et al. 2002; Herrick, Jun et al. 2002; Pasero, Bensimon et al. 2002; Lebofsky and Bensimon 2003; Jun, Herrick et al. 2004; Caburet, Conti et al. 2005; Herrick, Conti et al. 2005; Lebofsky and Bensimon 2005; Lebofsky, Heilig et al. 2006; Patel, Arcangioli et al. 2006; Rao, Conti et al. 2007; Schurra and Bensimon 2009; Nguyen, Walrafen et al. 2011; Cheeseman, Rouleau et al. 2012; Mahiet, Ergani et al. 2012; Tessereau, Buisson et al. 2013; Cheeseman, Ropars et al. 2014; Tessereau, Lesecque et al. 2014; Vasale, Boyar et al. 2015). The techniques of these references, specifically those pertaining or relating to molecular combing, are hereby incorporated by reference to the publications cited above. Bensimon, et al., U.S. Pat. No. 6,303,296 discloses DNA stretching procedures, Lebofsky, et al., WO 2008/028931 also discloses Molecular Combing procedures.

Stretching nucleic acid, extracted from any source (from virus, bacteria to human through plants . . . ), provides immobilized nucleic acids in linear and parallel strands and is preferably preformed with a controlled stretching factor on an appropriate surface (e.g., surface-treated glass slides). After stretching, it is possible to hybridize sequence-specific probes detectable for example by fluorescence microscopy (Lebofsky, Heilig et al. 2006). Thus, a particular sequence may be directly visualized on a single molecule level. The length of the fluorescent signals and/or their number, and their spacing on the slide provides a direct reading of the size and relative spacing of the probes.

Molecular combing is a technique enabling the direct visualization of individual nucleic acid molecules and has numerous applications for DNA structural such as physical mapping (Michalet, Ekong et al. 1997; Tessereau, Buisson et al. 2013; Cheeseman, Ropars et al. 2014) and detection of rearrangements including deletions and amplifications like in the Ca²⁺-activated neutral protease 3 gene involved in the tuberous sclerosis (Michalet, Ekong et al. 1997) and in the BRCA1 and BRCA2 genes that confer predisposition to the hereditary breast and ovarian cancer syndrome (Gad, Aurias et al. 2001; Gad, Caux-Moncoutier et al. 2002; Gad, Klinger et al. 2002; Gad, Bieche et al. 2003; Cheeseman, Rouleau et al. 2012). WO2014140788 A1 and WO2014140789 A1 disclose a method for detecting the amplifications of sequences in the BRCA1 locus and for the detection of breakpoints in rearranged genomic sequences, respectively. WO2013064895 A1 discloses for detecting genomic rearrangements in BRCA1 and BRCA2 genes at high resolution using Molecular Combing and for determining a predisposition to a disease or disorder associated with these rearrangements including predisposition to ovarian cancer or breast cancer.

Molecular Combing has also been successfully to determine the number of gene copies, for example in the trisomy 21 (Herrick, Michalet et al. 2000), to elucidate the organization of repeats regions such as human ribosomal DNA (Caburet, Conti et al. 2005), D4Z4 (Nguyen, Walrafen et al. 2011) and RNU2 arrays (Tessereau, Buisson et al. 2013; Tessereau, Lesecque et al. 2014; Tessereau, Leone et al. 2015) and to detect integration of exogenous DNA such as viral integration (Herrick, Conti et al. 2005; Conti, Herrick et al. 2007). WO 2010/035140 A1 discloses a method for analysis of D4Z4 tandem repeat arrays on human chromosomes 4 and 10 based on stretching of nucleic acid and on molecular combing.

Molecular Combing also applied to functional studies for the characterization of DNA replication (Herrick, Stanislawski et al. 2000; Herrick, Jun et al. 2002; Lebofsky and Bensimon 2003; Lebofsky and Bensimon 2005; Lebofsky, Heilig et al. 2006; Bailis, Luche et al. 2008; Daboussi, Courbet et al. 2008; Dorn, Chastain et al. 2009; Schurra and Bensimon 2009), DNA/protein interaction (Herrick and Bensimon 1999) and transcription (Gueroui, Place et al. 2002).

The patents referenced below describe various molecular combing procedures and individual steps useful in configuring a molecular combing procedure tailored to a particular purpose. Based on the present disclosure, those skilled in the art may adapt these procedures or their individual steps to detect, quantify or otherwise characterize genome or gene editing events performed by CRISPR-Cas9, other CRISPR-based or other genome or gene editing procedures.

One example of molecular combing from U.S. Pat. No. 6,303,296 comprises aligning a nucleic acid on a surface S of a support, wherein the process comprises: (a) providing a support having a surface S; (b) contacting the surface S with the nucleic acid; (c) anchoring the nucleic acid to the surface S; (d) contacting the surface S with a first solvent A; (e) contacting the first solvent A with a medium B to form an A/B interface, wherein said medium B is a gas or a second solvent; (f) forming a triple line S/A/B (meniscus) resulting from the contact between the first solvent A, the surface S, and the medium B; and (g) moving the meniscus to align the nucleic acid on the surface.

Another example, based on the disclosure of U.S. Pat. No. 7,985,542 comprises a method of detecting the presence of at least one domain of interest on a macromolecule to test that comprises: a) determining at least three target regions on the domain of interest, b) obtaining a corresponding labelled set of at least three probes each probe targeting one of said target region, the position of the probes one compared to the others being chosen and forming a sequence of at least two codes chosen between a group of at least two different codes, said sequence of codes being specific of the domain and being a specific signature of said domain of interest on the macromolecule to test; c) spreading the macromolecule and binding the probes to the macromolecule, wherein the spreading step occurs before or after the binding step, d) reading signals given by each of the labelled probes, each signal being associated with the label of said one probe, e) transcribing said signals in a sequence of codes established from the gap size between consecutive probes, f) detecting the sequence of codes of a domain of interest said sequence indicating the presence of said domain of interest on the macromolecule to test, and conversely the absence of detection of sequence of codes or part of sequence of codes of a domain of interest indicating the absence of said domain or part of said domain of interest on the macromolecule to test.

A third example of molecular combing based on the disclosure of U.S. Pat. No. 7,732,143 comprises a method of identifying a genetic abnormality comprising a break in a genome, wherein the method comprises: (a) providing a surface on which genomic DNA comprising a plurality of clones has been aligned using a molecular combing technique; (b) contacting the genomic DNA with at least one probe that is specific for a genomic sequence for which the genetic abnormality is sought; (c) detecting a hybridization signal between the at least one probe and the genomic DNA; (d) identifying the presence of the break in the genome directly or by comparing the length of the sequences detected by the hybridization signal to the length of sequences detected by a hybridization signal obtained using a control genome that does not contain the break and the at least one probe of part (b), and (e) determining the number of clones having a defined probe length, wherein the determined numbers of clones and the lengths of the sequences detected by the hybridization signals are converted into a graph.

In some embodiments of this method molecular combing, denaturation and hybridization involves one or more of the following experimental procedures.

Molecular Combing.

For analysis of human genome, a silanized coverslip is soaked in a disposable combing reservoir containing a solution of genomic DNA (3 μg/ml in 500 mM MES, pH 5.5), incubated at RT for 5 min then the coverslip is extracted from the reservoir using a molecular combing system. During the incubation, the DNA molecules become anchored on the surface through interaction between their extremities and hydrophobic surface. By extracting the surface from the reservoir, the interface between air and DNA solution moves relative to the surface and exerts a constant pulling force on the molecules remaining in the reservoir while the part of DNA exposed to air is progressively fixed onto the surface as an irreversible manner. The coverslips with combed DNA are then examined with an epifluorescence microscope so as to check the combing characteristics if necessary. The coverslips are then heated 4 hours at 60° C. They can be stored for several months if they are protected from moisture at −20° C. The coverslips dehydrated before denaturation procedure hereafter in a series of baths containing increasing concentrations of ethanol (70%, 90%, 100%).

Denaturation and Hybridization.

For probe preparation for each coverslip, 3-5 ng/kb de probes labeled with biotin, digoxigenin and/or Fluorescein are mixed with 5-10 μg of human DNA Cot1 and 10 μg of herring sperm DNA in 20 μl of hybridization buffer (50% deionized formamidell_2×SSC pH8.0, 0.5% sarkozyl, 0.5% SDS and 30% of BlockAid blocking solution (Thermofisher)). The probe solution is deposited on a clean glass slide, then the combed DNA coverslip is set on the droplet of probe solution (the probe solution is sandwiched between two glass surfaces). The slide is placed on hybridizer (Dako), to denature at 90° C. for 5 min then incubated at 37° C. overnight.

Immunodetection of hybridized probes. After hybridization, the coverslip is carefully removed from slide for washing three times in 2×SSC pH8.0 at 60° C. for 5 min each. Immunodetection solution (200 μL for one slide) is composed of 4 ng/μL BV480 Streptavidin (BD Bioscience), 70 ng/μL of each of Alexa Fluor 647 conjugated IgG Fraction Mouse Anti-Digoxin and Cy3 IgG Fraction Monoclonal Mouse Anti-Fluorescein (Jackson Immunoresearch) in BlockAid Blocking solution (ThermoFisher). The immunodetection solution is deposited on a clean glass slide, then the hybridized side of coverslip is set on the droplet. The slide is incubated at 37° C. for 30 min in a humidity chamber. After incubation, the coverslip is carefully removed from slide for washing three times in 2×SSC with 1% Tween 20 for 5 min each at ambient temperature. The coverslip is washed once in 1×PBS for 5 min followed by dehydration in a series of ethanol bath (70, 90, and 100%) for 1 min each. The coverslip can be stored for a couple of day at 4° C. under protection from light.

None of these patents referenced above contemplated using molecular combing in combination with CRISPR-Cas9 like genomic or gene editing or the advantages attained by this combination including the avoidance of bias and the improved efficiency provided by a single assay as disclosed herein.

The inventors disclose herein the tool in the context of probe pattern design for characterization of specific loci of interest with molecular combing technology. The constraints encountered when designing probe patterns are twofold: (i) The presence of segmental duplications and repeat elements can create signals that bias the analysis of the regions of interest (ROIs); and (ii) DNA breakage during the extraction step can render localization of partial signals problematic.

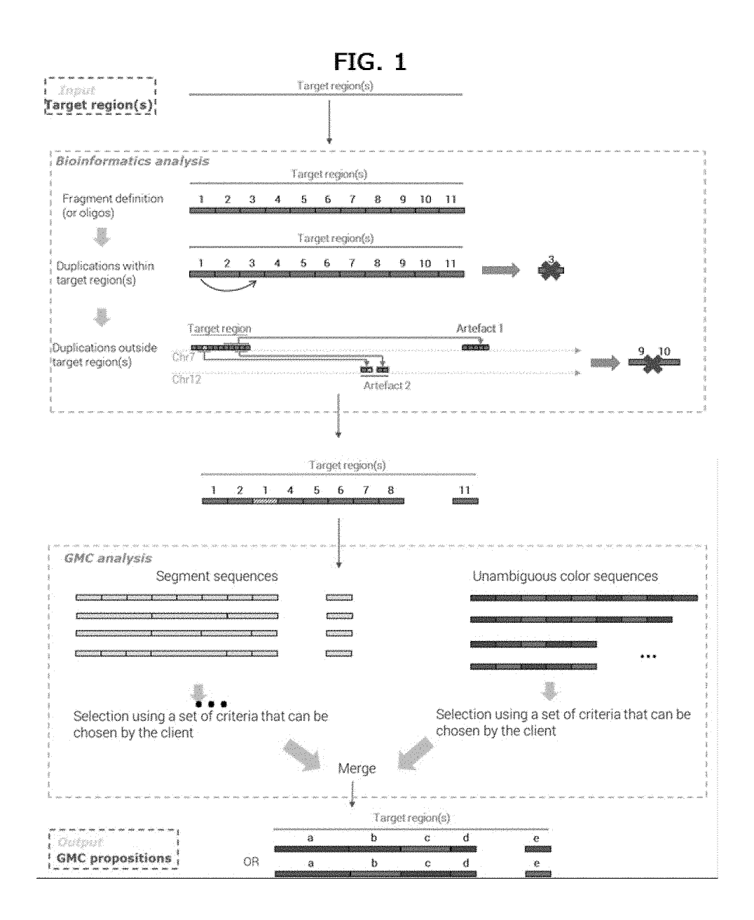

The method presented here tackles both technical issues by linking bioinformatics and combinatorial in silico analysis. The overall pipeline of the algorithm is described in FIG. 1 which depicts the overall scheme for design tool for color-coded GMCs. It takes as input either the sequence or the genomic coordinates of the targeted region, or multiple targeted regions, and returns a list of propositions for color-coded probe patterns for each region.

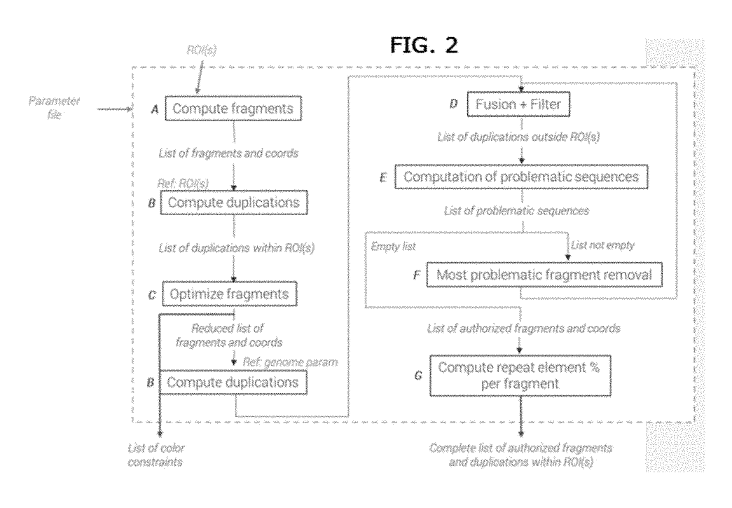

The first part of the algorithm, which workflow is detailed in FIG. 2, performs bioinformatics analysis of the genetic regions of interest. The bioinformatics part of the algorithm is composed of the following sections:

(A) The algorithm separates the regions into smaller fragments of the same size, which value is specified by a parameter. Depending on the labelling technique applied, either genetic fragments of several kilobases, or oligonucleotide fragments of dozens of base pair can be defined. If specified, this step optimizes fragment definitions to avoid sequences rich in repeat elements from design using online data bases such as RepeatMasker; see Jurka, J, 2000; Smit A F A, 1996-2010, each incorporated by reference. The constraints of feasibility for synthesis or amplification of the resulting fragments are not considered. Specific constraints of fragment definition can be specified in input, such as imposing coordinates for some fragments or imposing a subregion without fragment coverage.

(B) Segmental duplications are identified using a multiple sequence alignment algorithm such as BLAST, BLAT, FASTA, MUSCLE or CLUSTAL W; see Camacho et al., 2008, Kent, 2002, Pearson and Lipman, 1988, Edgar et al., 2004; Chenna et al. 2003; all of which are incorporated by reference. The last or any other publically or commercially available versions of any of these multiple sequence alignment programs prior to this application's filing date may be used. The BLAST algorithm is currently implemented in our method, as a performance study suggested its output was best adapted to the application of probe pattern design. However, any program based on multiple sequence alignment algorithms can be used instead. The alignment algorithm is launched successively on different reference sequences. First it is launched on the regions of interest for fragment optimization of region coverage (see step C). Then, it is launched, after step C, on the complete human genome (Rosenbloom, 2015) for identification of problematic segmental duplications in the genome outside of the regions of interest; see steps D to F.

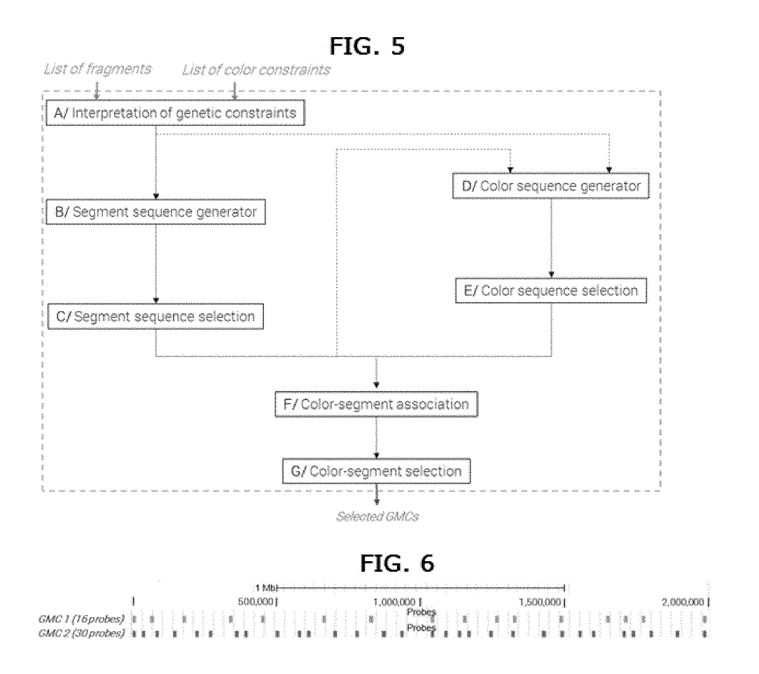

(C) The number of fragments to cover the region of interest is optimized from analysis of segmental duplications within the regions. A fragment that is almost entirely covered by duplications from other fragments of any of the regions of interest is removed. Moreover, this step identifies a list of color constraints emerging from the partial coverages of fragments by any other regions' fragments. This constraint list provides useful information for the combinatorial part of the algorithm, when colors are associated to fragments; see FIG. 5. The parameters defining the limits for fragment removal or definition of a color constraint are defined in Table 1.

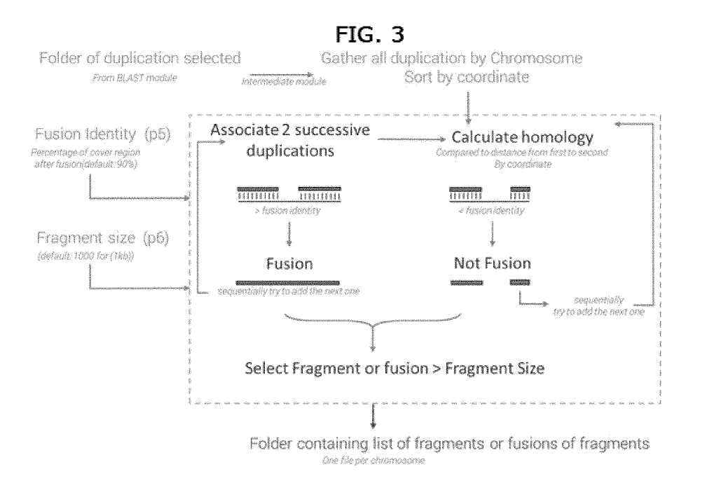

(D) This step post-processes results of genome alignment algorithm launched on the whole genome. The version of the reference genome to be used can be specified by a parameter defined in Table 1. Step D scans all resulting duplications and merges them when there are distanced by less than a proportion of the combination of their lengths; see FIG. 3 for details. The resulting duplications are then filtered by homology and length. The pipeline of this step is described in FIG. 3, and the default parameter values are listed in Table 1.

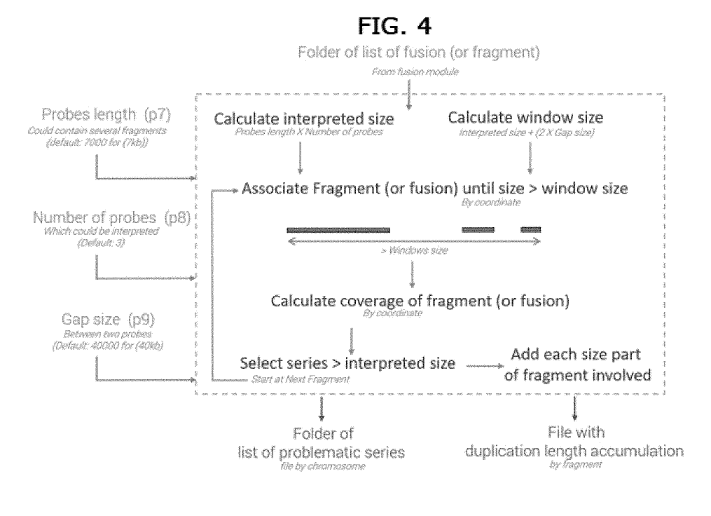

(E) This step identifies duplications that can create problematic sequences, i.e., that can create signals outside of the regions of interest, that can be misinterpreted as informative about said regions. In the case of probe pattern application to molecular combing technology, a problematic sequence is identified when, scanning the genome with a window of fixed size, a certain length of duplicated sequences is present in this window. The presence of overlap between the duplicated fragments is taken into account so that the overlap is not counted twice in the computation of duplication length. FIG. 4 describes the workflow and Table 1 the parameters for problematic sequence identification.

(F) When the removal of problematic sequences is required, fragments which duplicate in these problematic sequences are listed and sorted using a hand-defined score. The sorting score is computed for each fragment as the sum of lengths of its duplication occurrence in all problematic sequences. The fragments with the highest scores are successively removed from the region coverage up to the complete disappearance of problematic sequences.

Finally, the bioinformatics part of the design tool returns a list of fragments to be labelled that guarantees the absence of signal pollution due to genetic specificity of the regions of interest, as well as a PDF report containing graphical representation of ROI(s) coverage and excluded fragments.

As mentioned above, it is possible to constrain fragment definition by specifying coordinates in step (A). Moreover, it is also possible to impose that these fragments are not removed during computation and are maintained in the final list. In this case, these fragments are excluded from fragment removal of steps (C) and (F). When the constraints of fragment presence prevent complete removal of problematic sequences, a warning is published and the remaining problematic sequences are listed in the PDF report.

TABLE 1
List of parameters for bioinformatics part of the algorithm.

| | Tool | | | |
|---|---|---|---|---|
| | section | Default | | |
| Name | impacted | value | | Description |
| Fragment size | A | 2000 | (bp) | Definition of fragment size |
| Minimum | A | 500 | (bp) | Minimal size for fragment definition (when constraints |
| fragment_size | | | | prevent to define « fragment_size » fragments) |
| Genome | B | hg38 | | Specifies which reference genome version to use |
| Homology | B | 90 | (%) | Minimal percentage of alignment similarity in order to |
| | | | | consider duplication |
| Coverage | C | 90 | (%) | Minimal percentage of fragment coverage by |
| | | | | duplication of other fragments to remove inside ROI |
| | | | | fragment |
| Minimal | C | 40 | (%) | Minimal percentage of fragment coverage by |
| coverage | | | | duplication of other fragments to define a color |
| | | | | constraint |
| Fusion identity | D | 90 | (%) | Minimal percentage of similarity to merge fragments |
| Fusion | D | 1000 | (bp) | Minimum size for selection of fusion fragments |
| fragment size | | | | |
| Probe length | E | 7000 | (bp) | Length of probes for computation of problematic |
| | | | | sequences |
| Number of | E | 3 | | Number of probes for computation of problematic |
| probes | | | | sequences |
| Gap size | E | 40000 | (bp) | Gap size between two probes for computation of |
| | | | | problematic sequences |

| | | | |
|---|---|---|---|
| Repeat type | A, G | ALU | Type of repeat elements to consider for computation of |
| | | | repeat element percentage (RepeatMasker annotations) |

The inventors contemplate and disclose the following improvements.

In the case were problematic sequences that were not removed due to fragment presence constraints, the algorithm will, if required, add fragments close to these sequences in order to still be able to differentiate between signals of the ROIs and signals created by such sequences. Indeed, duplicated sequences outside the target genomic region will then be specifically labelled during the probe pattern design process in order to differentiate them during downstream analysis.

Division of the region into fragments (step A) can be performed so as to avoid presence of tandem repeats and inverted repeats within each fragment. To do so, the analysis of distribution of tandem repeats and inverted repeats in a fragment will be done using algorithm such as Tandem Repeat Finder and Inverted Repeat Finder (Benson, G. 1999; Warburton et al., 2004). Consequently, it will also be possible, when required by the sequences of the ROIs, to divide the region into fragments of distinct sizes.

The second part of the algorithm designs a color-coded probe pattern with a unique color pattern. In other terms, it transforms a list of fragments that can be labeled (and a set of constraints on labeling colors of these fragments) into a sequence of segments, each segment associated to a specific labelling color and composed of one or several fragments. The unique color coding allows a non-ambiguous localization of signals from the regions of interest, whether or not the probe pattern is fragmented by DNA breakage during sample preparation. The uniqueness of a partial pattern depends on total size of ROIs and representative length of prepared sample DNA. Longer ROIs require more complexity (e.g., a larger number of color segments) in given partial design, while the practical maximum degree of complexity is limited by the actual size of prepared DNA sample. The design process of segment patterns must then take ROIs and prepared DNA lengths into account, as well as lengths of segments and distances between segments. The length of the colored segments is constrained in order to guarantee efficient visualization, with the constraints being specific to the methodologies used for probe labelling and signal visualization. FIG. 5 describes the pipeline of the combinatorial part of the algorithm. Table 2 lists the parameters used for probe pattern design.

A priori knowledge about the biological phenomena investigated on the ROI(s) may hinder the design of the probe patterns. For example, one may be interested in detecting the presence of a characterized large rearrangement. In that case, an optimal probe pattern may include a set of segments beginning and ending at the exact positions of the rearrangement breakpoints. It is thus necessary to allow a flexible definition of probe pattern optimality. Table 3 lists the types of fragment-specific constraints that can be imposed on the design and Table 4 lists all the criteria that can be used for selecting sequences along the design process.

The algorithm is composed of the following sections:

(A) This subpart defines for each ROI a sequence of fragments and gaps, each associated with a name and a length. Gaps are defined when the distance between two consecutive fragments is longer than a parameter value C1 (see Table 2).

(B) All possible combinations of consecutive fragments over the regions of interest are generated. Each combination is called a “segment” and every segment is defined based on the following rules:
Segment length lies within an interval given by parameter value C2; see Table 2.
A segment cannot contain gaps.
   Moreover, specific combinations of fragments can be imposed; see Table 3.

(Bbis) Sequences of segments are generated from the list of all possible segments. The distributions of segments along the ROIs are defined by constraints on distance values between segments, on authorized minimal coverage of the ROIs and on acceptable amount of repeat elements per segment; see parameters C3, C4 and C5 of Table 2, respectively.

(C) The defined segment sequences are sorted and a selection is made based on a set of available criteria presented in Table 4. It is possible to combine a set of criteria and to impose priority levels to each of them. Thus, the algorithm provides a flexible definition of “optimal” probe pattern, which can be adjusted according to the type of experimental protocol used or the scientific question investigated.

(D) Color patterns are defined in this section such that any color subpattern above a minimal size; see parameter C6, Table 2; and its reverse subpattern have unique occurrences in the global set of color patterns. The list of available colors can also be specified, without any limit on the maximum number of colors; see parameter C7 of Table 2.

(E) Color patterns are associated with segment sequences such that each resulting probe pattern is defined by a set of fragments gathered in segments, each associated to a labelling color.

(F) This section selects the resulting probe patterns that respect the color constraints identified by the bioinformatics part of the algorithm (see Table 3).

At the end, the algorithm returns a list of colored segments, with genomic coordinates for each segment as well as their fragment composition. The composition of successive colored reagents provides a unique signature for detection of the presence, absence or modification of targeted regions. Moreover, subparts of this sequence of successive colored elements are also uniquely defined and enable the exact localization of partial or complete color-coded compositions.

TABLE 2
List of parameters for combinatorial part of the algorithm.

| | Tool | | |
|---|---|---|---|
| | section | | |
| Name | impacted | Description | Comment |
| C1 | A | Minimum distance | Can be defined differently |
| | | between two fragments | for each region of interest |
| | | to be considered as a | |
| | | significant gap | |
| C2 | B | Segment length interval | Can be defined differently |
| | | | for each region of interest |
| C3 | Bbis | Size interval of gaps | Can be defined differently |
| | | between two segments | for each region of interest |
| C4 | Bbis | Minimum coverage | Can be defined differently |
| | | percentage of the ROI | for each region of interest |
| | | authorized | |
| C5 | Bbis | Interval of authorized | Can be defined differently |
| | | percentage of repeat | for each region of interest. |
| | | element for every | |
| | | segment | |
| C6 | D | Minimal size of segment | Can be defined differently |
| | | sequences (in number of | for each region of interest |
| | | segments) for which a | |
| | | unique color pattern is | |
| | | required | |
| C7 | D | List of available colors | |

TABLE 3
List of types of constraints that can be imposed on the design of
segments and colors in the combinatorial part of the algorithm

| | Tool |
|---|---|
| | section |
| Constraints | impacted |
| Two fragments must be part of the same segment | B |
| Two fragments should not be part of the same | B |
| segment | |
| Two fragments should be attributed to the same | F |
| color (constraints identified by the bioinformatics | |
| part of the algorithm) | |
| A list of fragment must be part of gap | B |
| The color of one or several fragments can be | F |
| imposed | |

TABLE 4
Lists of criteria implemented for probe pattern selection
in the combinatorial part of the algorithm.

| | Tool |
|---|---|
| | section |
| Selection criteria | impacted |
| Favoring sequences with minimum (or maximum) number of | C |
| segments (either by ROI or in total) | |
| Minimizing length (in kilobases) of segment sequences for | C |
| which a unique color pattern is required (see C6 of Table 1) | |
| Maximizing (or minimizing) probe coverage of the ROI | C |
| Minimizing the overall percentage of repeat elements in | C |
| segments | |

The inventors contemplate and disclose the following features and/or improvements.

Definition of distinct parameter values of segment size interval (i.e., C2 of Table 2) according to the position of the segment along the regions of interest. Intake of information about fragment duplications within the ROIs will be taken into account in the generation and selection of color patterns and resulting probe patterns (sections D to F). When a priori knowledge of the characterization of large rearrangement of interest is available, it will not only be possible to take it into account for segment definition (step B, see Table 3) but also for color pattern generation (step D). Consequently, the color patterns generated by the large rearrangement will also have unique occurrences in the ROIs.

In the context of localization of replication kinetics on a ROI, the workflow of the combinatorial part of the algorithm for probe pattern design is slightly modified. For this specific assay, the recognition of subpatterns is not based on color patterns but on length patterns of gaps between segments; see Lebofsky, 2006. Thus, section D does not take a list of available colors as parameter (C7 of Table 2) but instead a list of gap lengths that are sufficiently distinct from each other that they will be easily identifiable on experimental signals resulting from molecular combing technology.

Gene or Genome Editing.

The GMC probes and methods disclosed herein are advantageously applied to analysis and detection of nucleic acid modifications produced by gene or genome editing procedures or to detecting non-damaged, damaged, or repaired nucleic acids. Representative, but not limited gene and genome editing procedures as described below.

Repair of DNA double strand breaks may be evaluated using the GMC probes and methods of the invention. Double strand breaks (DSB) in DNA are common events in eukaryotic cells that may induce deleterious damages and subsequently to genome instability and/or cell death. These events are typically repaired through either non-homologous end-joining (NHEJ) or homologous recombination (HR) pathways (Takata, Sasaki et al. 1998). The GMC probe(s) and methods disclosed herein are advantageously used in methods for detecting, analyzing or quantifying modifications to nucleic acids, such as genomic DNA, caused by DSBs.

Genome editing by NHEJ generally results in small deletions and/or insertions (indels) at the site of the break. NHEJ is an error prone mechanism that functions to repair DSBs without a template through direct relegation of the cleaved ends. This can create a frameshift mutation that may knockout gene function by a combination of two mechanisms: premature truncation of the encoded protein and non-sense-mediated decay of the mRNA transcript. NHEJ can occur during any phase of the cell cycle. In higher eukaryotes, NHEJ, rather than HR, is the dominant DSB repair system (Bibikova, Golic et al. 2002; Puchta 2005; Lieber 2010; Lieber and Wilson 2010).

HR relies on strand invasion of the broken end into a homologous sequence and subsequent repair of the break in a template-dependent manner (Szostak, Orr-Weaver et al. 1983). HR can be mediated by four different conservative and non-conservative mechanisms:

Gene conversion (GC). GC is basically initiated by the DSB formation at the recombination-recipient sites. The DSB ends are processed to have single stranded DNA tails, one of which eventually invades into the duplex of unbroken DNA. The invaded single strand DNA tail then forms a heteroduplex with the homologous DNA stretch in the unbroken template strand. The free DNA end of this heteroduplex primes a repair DNA synthesis. After a strand extension, the newly synthesized strand dissociates form the unbroken template DNA and anneals with the original broken DNA. Finally, the single strand DNA gap is filled followed by a ligation of DNA nicks. In this process, the DNA sequence on the unbroken DNA strand is converted to the broken strand, thereby accompanying a unidirectional transfer of genetic information (Paques and Haber 1999; Allers and Lichten 2001; Allers and Lichten 2001).

Non-allelic homologous recombination (NAHR). Indeed, HR can also occur ectopically between highly similar duplicated sequences or paralogous genomic segments, such as segmental duplications, through NAHR mechanism. NAHR can occur between directly oriented duplicated sequences on the same chromosome giving rise to a chromosomal deletion, and, if it occurs in an intermolecular fashion, it can generate a reciprocal duplication on the other chromosome. When NAHR takes place between duplicated sequences in an inverted orientation, it leads to inversions. NAHR is a mechanism leading to genomic variations and genomic disorders.

Break-induced replication (BIR). BIR pathway is employed to repair a DSB when homology is restricted to one end. In that case, recombination is used to establish a unidirectional replication fork that can copy the donor template to the end of the chromosome (McEachern and Haber 2006; Llorente, Smith et al. 2008). BIR mechanism is responsible of some segmental duplications (Payen, Koszul et al. 2008), deletions, nonreciprocal translocations, and complex rearrangements seen in a number of human diseases and cancers (Hastings, Lupski et al. 2009).

Single strand annealing (SSA). SSA is restricted to repair of DNA breaks that are flanked by direct repeats that can be as short as 30 nucleotides (Sugawara, Ira et al. 2000; Villarreal, Lee et al. 2012). Resection exposes the complementary strands of homologous sequences, which recombine resulting in a deletion containing a single copy of the repeated sequences through removal of the non-homologous single-stranded tails by the Rad1-Rad10 endonuclease complex (XPF-ERCC1 in mammals). SSA is therefore considered to be highly mutagenic.

When an exogenous DNA donor that has homologous sequences flanking the DSB is introduced along with the modified nuclease, the cell's machinery will use the supplied donor sequence as template for repair, thereby creating precise nucleotide change at or near the DSB site (Rouet, Smih et al. 1994). The length of the homologous region may vary between 70 to several hundred base pairs according to the nature of the donor DNA (single-stranded oligonucleotides or plasmids) (Yang, Guell et al. 2013; Hendel, Kildebeck et al. 2014). The donor DNA can be used to introduce either precise nucleotide substitutions or deletions, endogenous gene labelling, and targeted gene addition (McMahon, Randar et al. 2012). It has been shown that efficiency of gene targeting through HR in mammalian cells is stimulated by several orders of magnitude by introduction of DSB at the target site (Rouet, Smih et al. 1994; Choulika, Perrin et al. 1995; Smih, Rouet et al. 1995).

Gene or Genome Editing.

Genome editing with engineered nucleases is a technology that allows targeted modifications of any genomic DNA sequences (Baker 2012). This technology relies on the activation of the endogenous cellular repair machinery by DNA DSB through HR or NHEJ mechanisms as described above. The GMC probe(s) and methods disclosed herein are advantageously used in methods for detecting, analyzing or quantifying modifications to nucleic acids, such as genomic DNA, resulting from genome editing including, but not limited to those using the nucleases described below.

Four major types of nucleases exist to create targeted DNA DSB at specific site: zinc-finger nucleases (ZFNs), transcription activator-like effector-nuclease (TALENs), meganucleases and the CRISPR/Cas9 system (For review, (Maeder and Gersbach 2016; Merkert and Martin 2016).

Zinc finger nucleases. The zinc finger nuclease (ZFN)-based technology is based on the fact that the DNA-binding domain and the cleavage domain of the FokI restriction endonuclease function independently of each other (Li, Wu et al. 1992). Thus, chimeric nucleases with novel binding specificities can be produced by replacing the FokI DNA-binding domain with a zinc finger domain (Kim and Chandrasegaran 1994; Kim, Cha et al. 1996). Since ZFN-induced DSBs could be used to modify the genome through either NHEJ or HR (Bibikova, Carroll et al. 2001; Porteus and Baltimore 2003), this technology can be used to modify genes in both human somatic and pluripotent stem cell; see, each incorporated by reference.

TALENs.

The discovery of a simple one-to-one code dictating the DNA-binding specificity of TALE proteins from the plant pathogen Xanthomonas again raised the exciting possibility for modular design of novel DNA-binding proteins (Boch, Scholze et al. 2009; Moscou and Bogdanove 2009). The DNA binding domain contains a repeated highly conserved 33-34 amino acid sequence with divergent 12^{th} and 13^{th} amino acids. These two positions, referred to as the Repeat Variable Diresidue (RVD), are highly variable and show a strong correlation with specific nucleotide recognition. This relationship between amino acid sequence and DNA recognition allowed the selection of a combination of repeat segments containing the appropriate RVDs to target specific regions. This discovery of TALEs as a programmable DNA-binding domain was rapidly followed by the engineering of TALENs. Like ZFNs, TALEs were fused to the catalytic domain of the FokI endonuclease and shown to function as dimers to cleave their intended DNA target site (Christian, Cermak et al. 2010; Miller, Tan et al. 2011). Also similar to ZFNs, TALENs have been shown to efficiently induce both NHEJ and HR in human both somatic and pluripotent stem cells (For review, (Vasileva, Shuvalov et al. 2015; Merkert and Martin 2016).

Meganucleases.

Meganuclease technology involves re-engineering the DNA-binding specificity of naturally occurring homing endonucleases characterized by a large recognition site (double-stranded DNA sequences of 12 to 40 base pairs). There are currently six known families of meganucleases with conserved structural motifs: LAGLIDADG, HNH, His-Cys box, GYI-YIG, PD-(D/E)xk and Vsr-like families; see Belfort and Roberts, 1997, incorporated by reference. The largest class of homing endonucleases is the LAGLIDADG family, which includes the well-characterized and commonly used I-CreI and I-SceI enzymes (Cohen-Tannoudji, Robine et al. 1998; Chevalier and Stoddard 2001). Through a combination of rational design and selection, these homing endonucleases can be re-engineered to target novel sequences (Arnould, Perez et al. 2007; Grizot, Smith et al. 2009) and showed promise for the use of meganucleases in genome editing (Redondo, Prieto et al. 2008; Dupuy, Valton et al. 2013).

CRISPR/Cas9 system.

CRISPR-Cas RNA-guided nucleases are derived from an adaptive immune system that evolved in bacteria to defend against invading plasmids and viruses (Barrangou, Fremaux et al. 2007). Six major types of CRISPR system have been identified from different organisms (types I-VI) with various subtypes in each major type (Chylinski, Makarova et al. 2014; Makarova, Wolf et al. 2015). Within the type II CRISPR system, several species of Cas9 have been characterized from Streptococcus (S.) pyogenes, S. thermophilus, Neisseria meningitidis, S. aureus and Francisella novicida, so far (Gasiunas, Barrangou et al. 2012; Jinek, Chylinski et al. 2012; Mali, Aach et al. 2013; Sampson, Saroj et al. 2013; Zhang, Heidrich et al. 2013; Ran, Cong et al. 2015; Hirano, Gootenberg et al. 2016).

Three components are required for the CRISPR nuclease system to dictate specificity of DNA cleavage through Watson-Crick base pairing between nucleic acids: the CRISPR-associated (Cas) 9 protein, the mature CRISPR RNAs (crRNA) and a trans-activating crRNAs (tracrRNA) (Deltcheva, Chylinski et al. 2011). It has been showed that this system could be reduced to two components by fusion of the crRNA and tracrRNA into a single guide RNA (gRNA) (Jinek, Chylinski et al. 2012). To search for a DNA target, Cas9 nuclease only requires a 20-nucleotide sequence on the gRNA that base pairs with the target DNA and a DNA protospacer adjacent motif (PAM) adjacent to the complementary sequence (Marraffini and Sontheimer 2010; Jinek, Chylinski et al. 2012). Furthermore, re-targeting of the Cas9/gRNA complex to new sites could be accomplished by altering the sequence of a short portion of the gRNA.

While most of the Cas9 have similar RNA-guided DNA binding DNA mechanism, they often have distinct PAM recognition motif(s) expanding the targetable genome sequence for gene editing and genome manipulation. Furthermore, some types of CRISPR system may exhibit different mechanisms. For example, the type CRISPR system from Pyrococcus furiosus uses a Cas complex for RNA-directed RNA cleavage that allows targeting and modulation of RNAs in cells (Hale, Zhao et al. 2009; Hale, Majumdar et al. 2012). Recently, it has been shown that the protein Cpf1 (type V) isolated from Prevotela and Francisella uses a short crRNA without a tracrRNA for RNA-guided DNA cleavage and Cpf1-mediated genome targeting is effective and specific, comparable with the S. pyogenes Cas9 (Zetsche, Gootenberg et al. 2015; Dong, Ren et al. 2016; Fonfara, Richter et al. 2016; Yamano, Nishimasu et al. 2016). Finally, the type VI-A CRISPR effector C2c2 from Leptotrichia shahii is a RNA-guided RNase that can be programmed to knock down specific mRNAs in bacterium (Abudayyeh, Gootenberg et al. 2016). This diversity in natural CRISPR/Cas Systems may provide a functionally diverse set of editing tools.

Variants of the Cas9 system have also been developed. For example, a mutant form, known as Cas9D10A, with only nickase activity that can cleave only one strand and, subsequently only activate HR pathway when provided with a homologous repair template (Cong, Ran et al. 2013). Cas9D10A can even enhance specificity of gene editing by using a pair of Cas9D10A that target each strand of DNA at adjacent sites (Ran, Hsu et al. 2013). A nuclease deficient Cas9 (dCas9) that still has the capability to bind DNA is used to sequence-specifically target any region of the genome without cleavage. Instead, by fusing with various effector domain, dCas9 can be used as a gene silencing or activation tool (Maeder, Linder et al. 2013) or as a visualization tool when fused with fluorescent protein (Chen and Huang 2014).

In contrast to ZNFs, TALENs and meganucleases that described above, the CRISPR/Cas system does not require the engineering of novel proteins for each DNA target site. New sites can be targeted, simply by altering the short region of the gRNA that dictates specificity. Additionally, because the Cas9 protein is not directly coupled to the gRNA, this system is highly amenable to multiplexing through the concurrent use of multiple gRNAs to induce DSBs at several loci. Thereafter, numerous works demonstrated that the CRISPR/Cas9 system, mainly derived from the type II CRISPR system isolated from S. pyogenes, could be engineered for efficient genetic modification in mammalian cells (Cho, Kim et al. 2013; Cong, Ran et al. 2013; Mali, Yang et al. 2013) and to generate transgenic or knock-out animal models, from worm to monkey. The two patents mentioned below describe CRISPR-Cas9 or similar genome or gene editing procedures as well as individual steps useful in these procedures. Based on the present disclosure, those skilled in the art may adapt these genome or gene editing procedures or their individual steps to modify or edit a target polynucleotide.

A representative, but not limited, CRISPR system includes that disclosed by Zhang, U.S. Pat. No. 8,795,965 comprising a method of altering expression of at least one gene product comprising introducing into a eukaryotic cell containing and expressing a DNA molecule having a target sequence and encoding the gene product an engineered, non-naturally occurring Clustered Regularly Interspaced Short Palindromic Repeats (CRISPR)—CRISPR associated (Cas) system comprising one or more vectors comprising: a) a first regulatory element operable in a eukaryotic cell operably linked to at least one nucleotide sequence encoding a CRISPR-Cas system guide RNA that hybridizes with the target sequence, and b) a second regulatory element operable in a eukaryotic cell operably linked to a nucleotide sequence encoding a Type-II Cas9 protein, wherein components (a) and (b) are located on same or different vectors of the system, wherein the guide RNA is comprised of a chimeric RNA and includes a guide sequence and a trans-activating cr (tracr) sequence, whereby the guide RNA targets the target sequence and the Cas9 protein cleaves the DNA molecule, whereby expression of the at least one gene product is altered; and, wherein the Cas9 protein and the guide RNA do not naturally occur together.

Another representative, not limited, system is described by Frendewey, et al., U.S. Pat. No. 9,288,208 and comprises an in vitro method for modifying a genome at a genomic locus of interest in a mouse ES cell, comprising: contacting the mouse ES cell with a Cas9 protein, a CRISPR RNA that hybridizes to a CRISPR target sequence at the genomic locus of interest, a tracrRNA, and a large targeting vector (LTVEC) that is at least 10 kb in size and comprises an insert nucleic acid flanked by: (i) a 5′ homology arm that is homologous to a 5′ target sequence at the genomic locus of interest; and (ii) a 3′ homology arm that is homologous to a 3′ target sequence at the genomic locus of interest, wherein following contacting the mouse ES cell with the Cas9 protein, the CRISPR RNA, and the tracrRNA in the presence of the LTVEC, the genome of the mouse ES cell is modified to comprise a targeted genetic modification comprising deletion of a region of the genomic locus of interest wherein the deletion is at least 30 kb and/or insertion of the insert nucleic acid at the genomic locus of interest wherein the insertion is at least 30 kb. Other representative, but not limited, systems are described by WO 2014/089541 which is incorporated by reference and comprises methods for treating or repairing genes associated with hemophilia A. The methods of the present invention, which identify or quantify, corrections or repairs to genes are particular useful when used in conjunction with the genome or gene editing procedures described below because molecular combing easily detects genetic corrections and repaired genes provided made by these methods.

The F8 gene, located on the X chromosome, encodes a coagulation factor (Factor VIII) involved in the coagulation cascade that leads to clotting. Factor VIII is chiefly made by cells in the liver, and circulates in the bloodstream in an inactive form, bound to von Willebrand factor. Upon injury, FVIII is activated. The activated protein (FVIIIa) interacts with coagulation factor IX, leading to clotting. Mutations in the F8 gene cause hemophilia A (HA). Over 2,100 mutations in this gene have been identified, including point mutations, deletions, and insertion. One of the most common mutations includes inversion of intron 22, which leads to a severe type of HA. Mutations in F8 can lead to the production of an abnormally functioning FVIII protein or a reduced or absent amount of circulating FVIII protein, leading to the reduction of or absence of the ability to clot in response to injury. In one aspect, the present invention is directed to the targeting and repair of F8 gene mutations in a subject suffering from hemophilia A using the methods described herein. Approximately 98% of patients with a diagnosis of hemophilia A are found to have a mutation in the F8 gene (i.e., intron 1 and 22 inversions, point mutations, insertions, and deletions). Such a method may comprise introducing into a cell of the subject one or more isolated nucleic acids encoding a nuclease that targets a portion of an F8 gene containing a mutation that causes hemophilia A, wherein the nuclease creates a double stranded break in the F8 gene; and an isolated nucleic acid comprising a donor sequence comprising (i) a nucleic acid encoding a truncated FVIII polypeptide or (ii) a native F8 3′ splice acceptor site operably linked to a nucleic acid encoding a truncated FVIII polypeptide, wherein the nucleic acid comprising the (i) nucleic acid encoding a truncated FVIII polypeptide or (ii) native F8 3′ splice acceptor site operably linked to a nucleic acid encoding a truncated FVIII polypeptide is flanked by nucleic acid sequences homologous to the nucleic acid sequences upstream and downstream of the double stranded break in the DNA, and wherein the resultant repaired gene, upon expression, confers improved coagulation functionality to the encoded FVIII protein of the subject compared to the non-repaired F8 gene. Such a method may also involve inducing immune tolerance to a FVIII replacement product ((r)FVIII) in a subject having a FVIII deficiency and who will be administered, is being administered, or has been administered a (r)FVIII product comprising introducing into a cell of the subject one or more nucleic acids encoding a nuclease that targets a portion of the F8 gene containing a mutation that causes hemophilia A, wherein the nuclease creates a double stranded break in the F8 gene; and an isolated nucleic acid comprising a donor sequence comprising (i) a nucleic acid encoding a truncated FVIII polypeptide or (ii) a native F8 3′ splice acceptor site operably linked to a nucleic acid encoding a truncated FVIII polypeptide, wherein the nucleic acid comprising the (i) nucleic acid encoding a truncated FVIII polypeptide or (ii) native F8 3′ splice acceptor site operably linked to a nucleic acid encoding a truncated FVIII polypeptide is flanked by nucleic acid sequences homologous to the nucleic acid sequences upstream and downstream of the double stranded break in the DNA, and wherein the repaired gene, upon expression, provides for the induction of immune tolerance to an administered replacement FVIII protein product. Either of these methods may employ a nuclease that is a zinc finger nuclease (ZFN), Transcription Activator-Like Effector Nuclease (TALEN), or a CRISPR (Clustered Regularly Interspaced Short Palindromic Repeats)-associated (Cas) nuclease. Both of these methods may use a nuclease that intron 22 of the F8 gene, that targets intron 1 of the F8 gene, that targets the exon 22/intron 22 junction, or that targets the exon 1/intron 1 junction. Either of these methods may target an F8 mutation that comprises a mutation that is an intron 22 inversion.

Computer-Implementation.

In some embodiments the algorithms disclosed herein are transcribed into software and implemented on a computer. For long or complex target regions or projects requiring design of a large number of GMC probes, it may not be feasible to select GMC probes manually or to analyze the resulting data manually, for example, to design GMC probes for Molecular Combing of complex regions of the genome and to analyze the resulting data. Computer implementation permits efficient and timely design of GMC probes as well as analysis of quantities of molecular combing data that it would not be feasible to analyze manually. The methods for automatic detection of fluorescent signals in molecular combing scanned images and for automatic analysis of molecular combing data for detection of large rearrangements have been described in Patents WO2017153848 (published in 2017) and WO2017153844 (published in 2017), respectively.

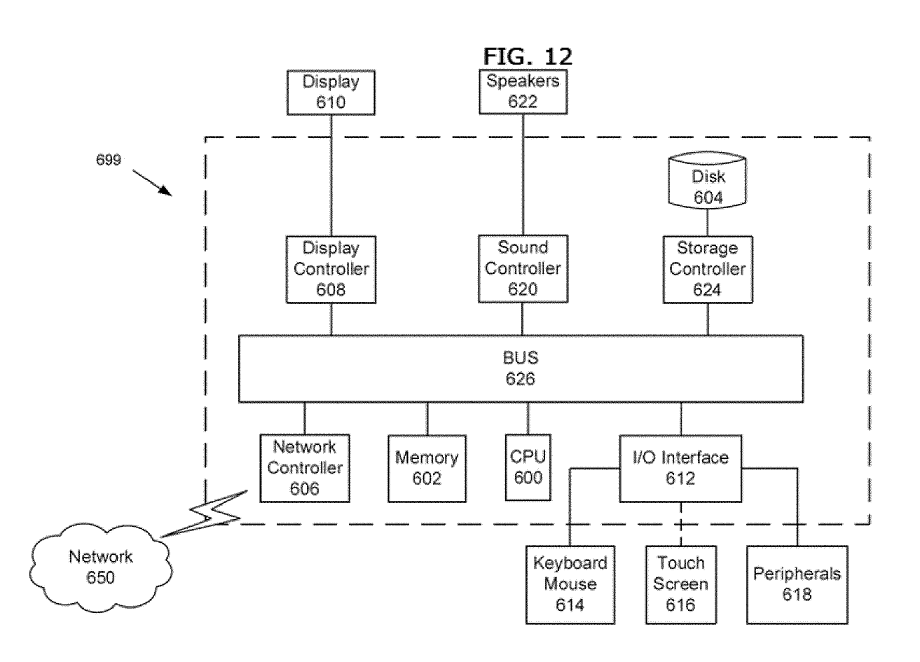

FIG. 12 illustrates a computer system upon which embodiments of the present disclosure may be implemented. Each of the functions of the above described embodiments may be implemented by circuitry, which includes one or more processing circuits. A processing circuit includes a particularly programmed processor, for example, processor (CPU) 600, as shown in FIG. 12. A processing circuit also includes devices such as an application specific integrated circuit (ASIC) and conventional circuit components arranged to perform the recited functions.

In FIG. 12, the device 699 includes a CPU 600 which performs the processes and implements the algorithms for design of GMC probes or for analyzing molecular combing data described above obtained from procedures using the GMC probes. The device 699 may be a general-purpose computer or a particular, special-purpose machine. In one embodiment, the device 699 becomes a particular, special-purpose machine when the processor 600 is programmed to participate in processing and analyzing molecular combing data, and/or perform one or more steps of the process of FIG. 12.

The process data and instructions may be stored in memory 602. These processes and instructions may also be stored on a storage medium disk 604 such as a hard drive (HDD) or portable storage medium or may be stored remotely. The instructions may be stored on CDs, DVDs, in FLASH memory, RAM, ROM, PROM, EPROM, EEPROM, hard disk or any other device with which the system communicates, such as a server or computer. In other words, the instructions may be stored on any non-transitory computer-readable storage medium to be executed on a computer.

Further, the discussed embodiments may be provided as a utility application, background daemon, or component of an operating system, or combination thereof, executing in conjunction with CPU 600 and an operating system such as, but not limited to, Microsoft Windows, UNIX, Solaris, LINUX, Android, Apple MAC-OS, Apple iOS and other systems known to those skilled in the art.

CPU 600 may be any type of processor that would be recognized by one of ordinary skill in the art. For example, CPU 600 may be a Xenon or Core processor from Intel of America or an Opteron processor from AMD of America. CPU 600 may be a processor having ARM architecture or any other type of architecture. CPU 600 may be any processor found in a mobile device (for example, cellular/smart phones, tablets, personal digital assistants (PDAs), or the like). CPU 600 may also be any processor found in musical instruments (for example, a musical keyboard or the like).

Additionally or alternatively, the CPU 600 may be implemented on an FPGA, ASIC, PLD or using discrete logic circuits, as one of ordinary skill in the art would recognize. Further, CPU 600 may be implemented as multiple processors cooperatively working in parallel to perform the instructions of the processes described herein.

The computer 699 in FIG. 12 also includes a network controller 606, such as, but not limited to, a network interface card, for interfacing with network 650. As can be appreciated, the network 650 can be a public network, such as, but not limited to, the Internet, or a private network such as an LAN or WAN network, or any combination thereof and can also include PSTN or ISDN sub-networks. The network 650 can also be wired, such as an Ethernet network, or can be wireless such as a cellular network including EDGE, 3G and 4G wireless cellular systems. The wireless network can also be WiFi, Bluetooth, or any other wireless form of communication that is known.

The computer 699 further includes a display controller 608, such as, but not limited to, a graphics adaptor for interfacing with display 610, such as, but not limited to, an LCD monitor. A general purpose I/O interface 612 interfaces with a keyboard and/or mouse 614 as well as a touch screen panel 616 on or separate from display 610. General purpose I/O interface also connects to a variety of peripherals 618 including printers and scanners. The peripheral elements discussed herein may be embodied by the peripherals 618 in the exemplary embodiments.

A sound controller 620 may also be provided in the computer 699 to interface with speakers/microphone 622 thereby providing sounds and/or music. The speakers/microphone 622 can also be used to accept dictated words as commands.

The general purpose storage controller 624 connects the storage medium disk 604 with communication bus 626, which may be an ISA, EISA, VESA, PCI, or similar. A description of the general features and functionality of the display 610, keyboard and/or mouse 614, as well as the display controller 608, storage controller 624, network controller 606, sound controller 620, and general purpose I/O interface 612 is omitted herein for brevity as these features are known.

The method of the invention cannot be performed without use of a computer, as some steps include using alignment algorithms such as BLAST. The search for duplicated sequences in complex genomes such as human or mouse genomes involves performing an immense number of complex operations on very long sequences (i.e. at least 1 megabases long) and thus cannot be performed manually.

It is possible to perform BLAST algorithms using publicly available websites and to then perform manually the other technical steps of the method of the invention. However, the automated method of the invention has several significant advantages over a manual process of every technical step described above. First, study of some target regions may imply design of long sequences of colored probes (up to 30 for example for localization of replication signals in a region of 2 MB, see example below and FIG. 6) or may imply designing probe sequences simultaneously for several target regions. In these cases, the design of a sequence of colors (or multiple color sequences) that ensures unicity of any partial sequence from a specified size is a complex task and requires mathematical operations that are much more efficiently computed automatically. Secondly, the automated method is more robust than a combination of manual operations. Indeed, it prevents human error during manual handling of data files such as upload and download of data files to and from webtool interfaces or manual modifications of data sheets. Moreover, human selection of alignment results and definition of duplicated subsequences can be subjective and human analysis is prone to different subject-specific biases or errors. With this innovative automated process, the parameters are fixed once and for all, and the results will be steady and comparable for any set of target regions at any time.

Finally, the automated method takes only few hours to be fully completed, whereas manual process of all technical steps can take days, depending on the quantity of duplicated sequences found outside of the regions of interest and on the size of the color sequence that has to be uniquely defined. The computation time of the automated method can still be greatly accelerated by the use of GPU optimized code or via a parallelization of the process on a network of linked computers on the cloud without any modification of the proposed method.

The automated method of the invention is also very much time-saving compared to the Genomic Morse Code approaches previously employed. Indeed, resulting GMC probes of the latter are not guaranteed to produce uniquely identifiable experimental signals and thus can produce uninterpretable results. Consequently, in such cases, experimental results obtained from GMC probes are not informative and a new design is needed with additional specific constraints (see example of study of HNPCC region described below). The automated method presented here enables to skip directly to second optimal design and save the whole time and resource effort of the first GMC Design and the first set of experiments that produced uninterpretable results.

### EXAMPLES

### Probe Pattern for Characterization of Replication Kinetics on a Region of 2 Megabases

Molecular combing assays for study of replication kinetics combine bi-color fluorescence signals created by replication events and a mono-color probe pattern based on a length code of spaces between probes enabling the localization of replication signals onto the region of interest [Lebofsky, 2006]. In that particular example, segmental duplications were excluded and two probe patterns with different numbers of probes (16 and 30, respectively) were computed. The parameters values of the bioinformatics part of the design algorithm were the values by default defined in Table 1, except for “Probe length” and “Gap size” parameters, which were set at 3000 bp and 200000 bp, respectively. These two parameters are used for the definition of duplicated sequences outside the target region that are problematic for downstream analysis of experimental signals. The parameter values were modified in order to mimic the particular characteristics of probe patterns used for localization of replication signals, i.e., the low probe density due to large gaps between probes. Moreover, a modified version of the combinatorial part of the design algorithm was used, so as to compute unique sequences of gap lengths instead of unique color-coded sequence. The gap values were fixed at either 20, 35, 50, 65, 80, 95, 110, 125, 140, 155, 170, 185 or 200 kb. FIG. 6 presents both mono-color probe patterns for localization of replication signals on a region of 2 megabases in chromosome 7. Tables 5 and 6 list all probe coordinates (relatively along the target region) and gap lengths of both probe patterns. The distances between fluorescent probes enable the reconstruction of the locus from molecular combing signals. Each signal containing at least 3 probes can be unambiguously localized onto the region of interest using patterns of gap values. Each probe measures 12 kb and each gap measures between 20 kb and 200 kb. See FIG. 6 which shows the relative positions of DNA probes to hybridize along the region of interest. The colors of the probes are graphical representations and do not limit the choice of colors for experimental process. Graphics were obtained using the Genome Browser webtool; see Genome Browser (2017).

TABLE 5
Relative coordinates of probes along the target region of 2 megabases
for the probe pattern containing 16 probes. The last column precises
the length in kilobases (kb) of the gap before each probe.

| | Begin probe | End probe | Gap length |
|---|---|---|---|
| Probe Id | coordinate | coordinate | (kb) |
| P1 | 0 | 12000 | 0 |
| P2 | 62000 | 74000 | 50 |
| P3 | 174000 | 186000 | 100 |
| P4 | 336000 | 348000 | 150 |
| P5 | 448000 | 460000 | 100 |
| P6 | 660000 | 672000 | 200 |
| P7 | 822000 | 834000 | 150 |
| P8 | 1034000 | 1046000 | 200 |
| P9 | 1146000 | 1158000 | 100 |
| P10 | 1258000 | 1270000 | 100 |
| P11 | 1320000 | 1332000 | 50 |
| P12 | 1482000 | 1494000 | 150 |
| P13 | 1644000 | 1656000 | 150 |
| P14 | 1706000 | 1718000 | 50 |
| P15 | 1768000 | 1780000 | 50 |
| P16 | 1980000 | 1992000 | 200 |

TABLE 6
Relative coordinates of probes along the target region of 2 megabases
for the probe pattern containing 30 probes. The last column precises
the length in kilobases (kb) of the gap before each probe.

| | Begin probe | End probe | Gap length |
|---|---|---|---|
| Probe Id | coordinate | coordinate | (kb) |
| P1 | 0 | 12000 | 0 |
| P2 | 32000 | 44000 | 20 |
| P3 | 79000 | 91000 | 35 |
| P4 | 141000 | 153000 | 50 |
| P5 | 218000 | 230000 | 65 |
| P6 | 265000 | 277000 | 35 |
| P7 | 357000 | 369000 | 80 |
| P8 | 389000 | 401000 | 20 |
| P9 | 496000 | 508000 | 95 |
| P10 | 573000 | 585000 | 65 |
| P11 | 605000 | 617000 | 20 |
| P12 | 697000 | 709000 | 80 |
| P13 | 774000 | 786000 | 65 |
| P14 | 866000 | 878000 | 80 |
| P15 | 928000 | 940000 | 50 |
| P16 | 1035000 | 1047000 | 95 |
| P17 | 1082000 | 1094000 | 35 |
| P18 | 1129000 | 1141000 | 35 |
| P19 | 1161000 | 1173000 | 20 |
| P20 | 1238000 | 1250000 | 65 |
| P21 | 1315000 | 1327000 | 65 |
| P22 | 1422000 | 1434000 | 95 |
| P23 | 1484000 | 1496000 | 50 |
| P24 | 1546000 | 1558000 | 50 |
| P25 | 1593000 | 1605000 | 35 |
| P26 | 1700000 | 1712000 | 95 |
| P27 | 1732000 | 1744000 | 20 |
| P28 | 1794000 | 1806000 | 50 |
| P29 | 1886000 | 1898000 | 80 |
| P30 | 1978000 | 1990000 | 80 |

Probe Pattern for Detection of Large Rearrangement in HNPCC Region.

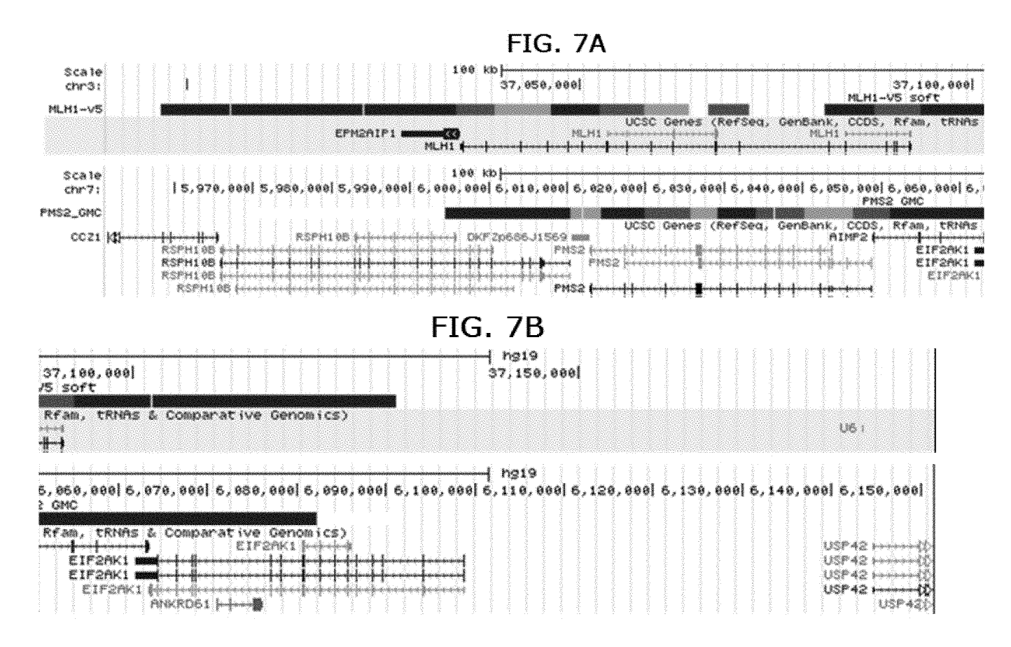

The GMC approach was applied to the study of large rearrangements in the regions containing 2 of the genes involved to hereditary nonpolyposis colon cancer (HNPCC): MLH1 and PMS2. A set of 2 probe patterns was designed based on the constraints described in the patent about a method for detecting large rearrangements; see Komatsu, 2016. These probe patterns are visible on the website of Genomic Vision (GV, 2016) and shown in FIG. 7A and FIG. 7B. Molecular combing experiments were produced with simultaneous hybridization of these probe patterns. It appeared during the downstream analysis of the experimental signals that the designed probe patterns were not optimal for the study of large rearrangement in both covered regions in the same experimental process. Indeed, due to DNA fragmentation during extraction step of molecular combing technology the experimental signals often only provided information about partial parts of the probe patterns. Yet, these probe patterns do not allow an efficient reconstitution of the region, with color and length information of probes, from partial signals.

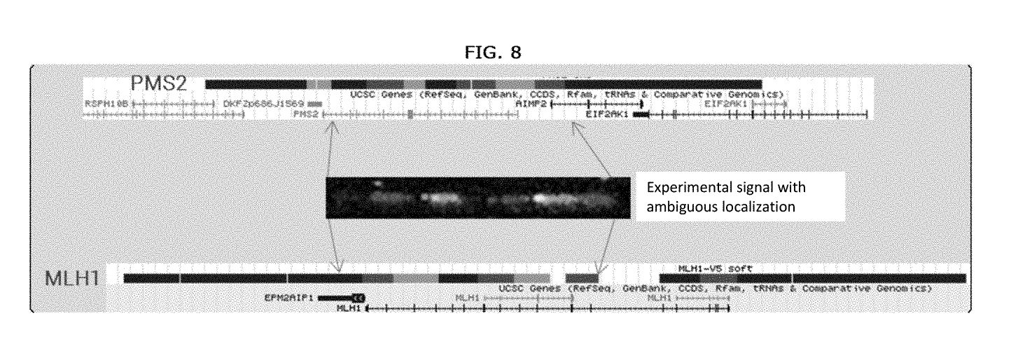

FIG. 8 shows the example of an experimental signal obtained by molecular combing and hybridization of both MLH1 and PMS2 probes on the same coverslips which color-pattern and length-pattern do not able us to determine which DNA region it comes from. Indeed, the signal of 40 kb length covers a pattern of 7 colored probes that could either correspond to a sub part of the PMS2 probe pattern (above the signal image in FIG. 8) or a sub part of MLH1 probe pattern (below the signal image in FIG. 8). This case of ambiguous color patterns is not isolated and similarly, 17 other partial probe patterns of variable lengths (from combinations of 3 to 8 probes) have several occurrences along the complete probe patterns. Consequently, experimental signals that only contain one of 18 ambiguous partial probe patterns mentioned above cannot be uniquely localized on the regions of interest, and have to be excluded from downstream analysis of the molecular combing experiments, thus reducing the information content of the set of experimental results.

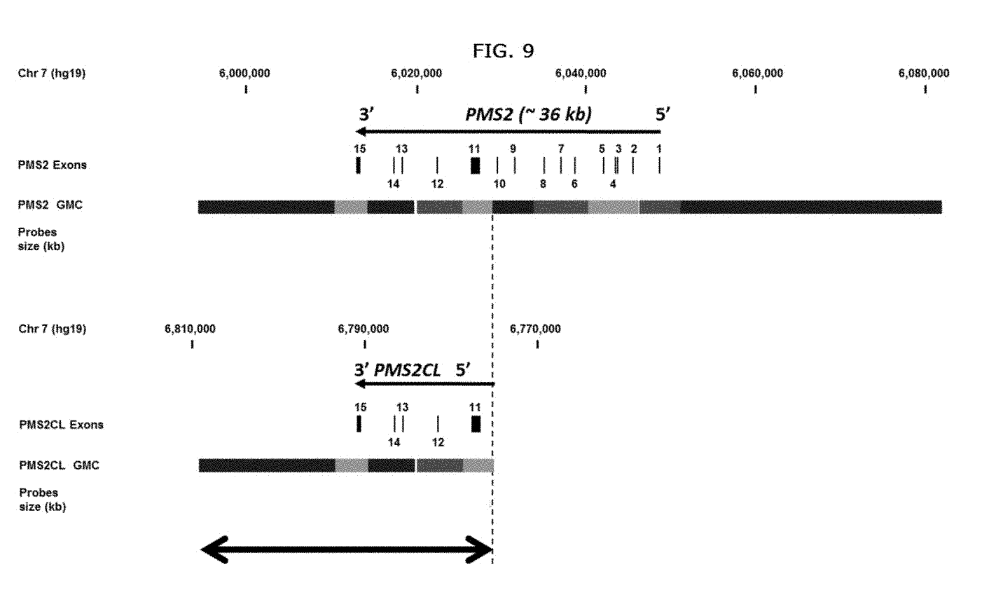

Moreover, it has been observed during downstream analysis that the first 35 kb of the probe pattern covering PMS2 gene also duplicate outside of the region of interest, 800,000 kilobases (kb) further on chromosome 7 according to GRCh19/hg19 human genome. This segmental duplication thus creates artefact experimental signals that emerge from the hybridization of PMS2 probes onto the duplicated sequence. FIG. 9 depicts this segmental duplication of the first 35 kb of the probe pattern covering PMS2 gene as well as the probe pattern created by the duplication. Consequently, any experimental signal containing at least 3 probes created by the duplicated region can be wrongly interpreted as originating from the region of interest. This ambiguity in the localization of experimental signals further complicates the possibility for correct interpretation of experimental data for the analysis of large rearrangements in targeted regions.

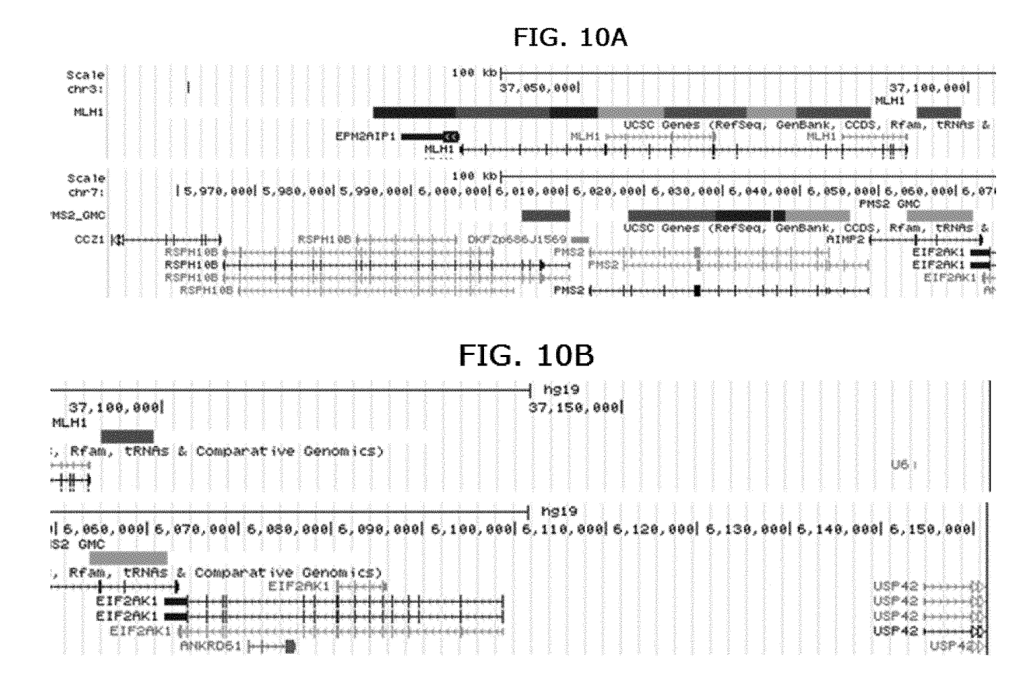

We show in FIG. 10 shows an example of probe patterns designed on the same regions of interest with the method for probe pattern design described in this document. Tables 7 and 8 list the probe coordinates, lengths and colors for MLH1 and PMS2 regions, respectively.

TABLE 7
Probe coordinates, lengths and colors for probe pattern
of MLH1 region in chromosome 3. Coordinates are reported
according to GRCh19/hg19 human genome.

| | Begin probe | End probe | Probe length | |
|---|---|---|---|---|
| Probe ID | coordinate | coordinate | (kb) | Color |
| P1 | 37023669 | 37034299 | 10,63 | Blue |
| P2 | 37034273 | 37046400 | 12,127 | Green |
| P3 | 37046376 | 37052529 | 6,153 | Blue |
| P4 | 37052516 | 37061090 | 8,574 | Green |
| P5 | 37060960 | 37071561 | 10,601 | Red |
| P6 | 37071545 | 37077986 | 6,441 | Green |
| P7 | 37077985 | 37087538 | 9,553 | Red |
| P8 | 37093415 | 37099185 | 5,77 | Red |

TABLE 8
Probe coordinates, lengths and colors for probe pattern
of PMS2 region in chromosome 7. Coordinates are reported
according to GRCh19/hg19 human genome.

| | Begin probe | End probe | Probe length | |
|---|---|---|---|---|
| Probe ID | coordinate | coordinate | (kb) | Probe color |
| P1 | 6004154 | 6010491 | 6,337 | Red |
| P2 | 6017868 | 6029101 | 11,233 | Red |
| P3 | 6029084 | 6038053 | 8,969 | Blue |
| P4 | 6038035 | 6046439 | 8,404 | Green |
| P5 | 6053673 | 6062591 | 8,918 | Green |

Each probe measures between 5.7 kb and 12.2 kb and gap lengths lie between 0 kb and 7.8 kb. The algorithmic part of the design algorithm described in this document has been launched on MLH1 and PMS2 genetic regions with the default parameter values defined in Table 1, and with the constraints of keeping the duplicated sequences outside of the region of interest. Indeed, in that case, we decided to keep the duplicated sequence of the first 35 kb of PMS2 although it was identified by the algorithm. However, during the combinatorial part of the design algorithm, we imposed the design of only 2 probes in the duplicated sequence using constraints on segment positions listed in Table 3. Moreover, we set the parameter C6 of Table 2 to the value of 3, the color list parameter C7 to be composed of colors blue, red and green, and we set the other parameters of Table 2 so as to influence at minima on the design of color probe patterns. The design method then guarantees that any experimental signal obtained from probe patterns defined in FIG. 10 and containing at least 3 probes provides unambiguous and relevant information for the analysis of large rearrangements. Indeed, it has been taken into account in the design that each color pattern of 3 probes is unique among the regions of interest. Moreover, the method accounted for the presence of the segmental duplication, forcing the duplicated region to contain at most only 2 probes.

As shown above, probe patterns designed based on the GMC approach created up to 24 types of experimental signal (containing patterns of 3 probes or more) that could be wrongly interpreted and bias large rearrangement study (18 due to multiple pattern occurrence in ROIs, 6 due to segmental duplication outside the ROI). The probe pattern approach described here guarantees that, with the new designed probe patterns, every experimental signal containing at least 3 probes can be unambiguously interpreted.

Probe Pattern for Characterization of the SMA Region.

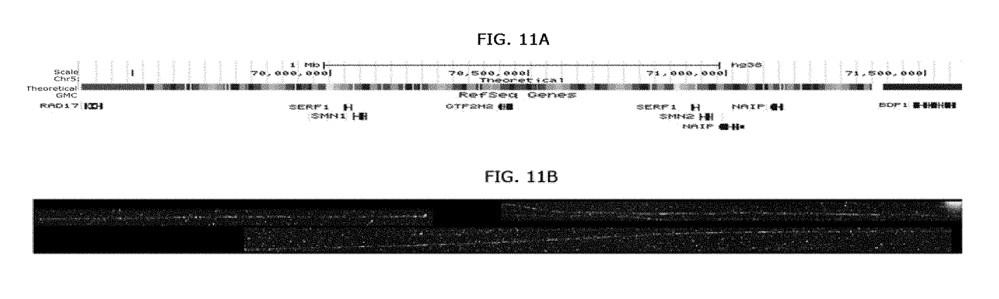

The locus for Spinal Muscular Atrophy (SMA locus) is a complex genetic region that contains a substantial amount of large segmental duplications that renders very difficult the reconstitution of the locus using sequencing methods. FIG. 11A presents a probe pattern computed for the characterization of the SMA locus using the design method described in this document. The design algorithm was launched using default parameter values for the bioinformatics part of the algorithm, as well as a constraint to keep duplicated sequences out of the region of interest. The last constraint was applied because the analytical method for the reconstitution of SMA locus only considers very long experimental signals (above 500 kb) and thus automatically excludes signals from duplicated sequences outside of the region of interest. For the combinatorial part of the algorithm, we set the color sequence parameter C7 to contain colors red, blue, green, magenta, yellow and cyan, and we set all other parameters of Table 2 so as to influence at minima on the design of color probe patterns. We also imposed the selection of probe patterns based on minimization of the length of segment sequences required to guarantee unique color coding (see Table 4). Probe lengths lie within 3 kb and 170 kb. FIG. 11A depicts the relative positions of DNA probes according to GRCh38/hg38 human genome (Rosenbloom et al., 2015). The relative position of genes localized on the SMA locus are indicated below the probe pattern. FIG. 11B presents examples of experimental signals obtained by molecular combing and hybridization of the probe pattern for SMA locus characterization. The signals are manually aligned with one another so as to reconstitute the full SMA probe pattern. Molecular combing experiments with that probe pattern enabled a new precise characterization of the SMA locus and the discovery of a non-registered CNV; Pierret et al., 2016.

Probe Pattern for Analysis of Large Rearrangements Between a Gene and its Pseudo-Genes.

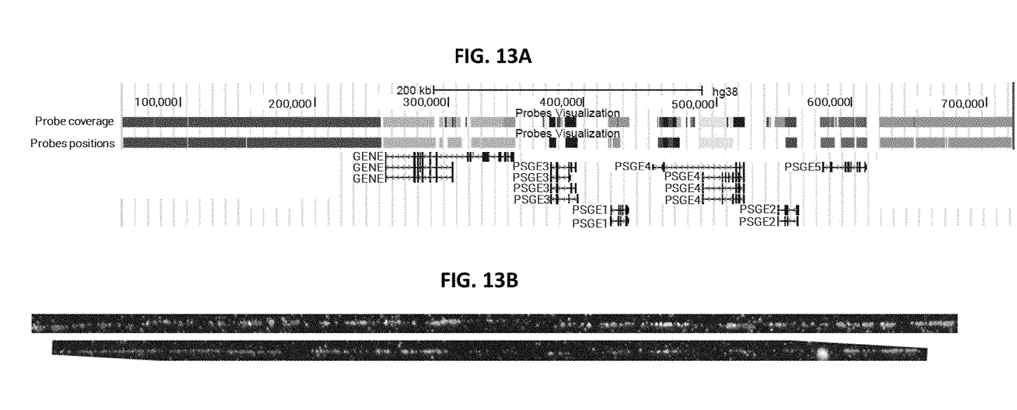

A probe pattern has been defined with the invention method for the study of all encountered rearrangements in a genetic region in chromosome 1 of human genome that contains a main gene and 5 pseudogenes which order and presence vary between individuals. The design algorithm was launched with default parameter values for the bioinformatics part of the algorithm and with constraints to remove probe fragments between gene and pseudo-gene positions. For the combinatorial part of the algorithm, we imposed when possible to have one probe segment or at least one color per gene or pseudo-gene, we set the color sequence parameter C7 to contain colors red, blue, green, magenta, yellow and cyan, and we set all other parameters of Table 2 so as to influence at minima on the design of color probe patterns. FIG. 13A presents the probe pattern computed for the analysis of large rearrangements between a gene and its 5 pseudo-genes. The color pattern of probes to be synthesized is shown as the below probe pattern called “Probe positions”. The resulting coverage of the region by the defined probes, that takes duplications within the region of interest into account, is shown as the above probe pattern called “Probe coverage”. Relative positions of DNA probes along the region of interest are specified. The relative positions of genes and pseudo-genes are localized on the target locus and indicated below the probe pattern. In the figure, “GENE” stands for the gene of interest and “PSGE1”, “PSGE2”, ‘PSGE3”, “PSGE4” and “PSGE5” for the 5 pseudo-genes of “GENE” gene. Graphical representations of the probe pattern were obtained using the Genome Browser webtool; see Genome Browser (2017). Table 9 lists the probe coordinates, lengths and colors for the chromosome 1 region of interest.

TABLE 9
Probe coordinates, lengths and colors for probe pattern
of target region in chromosome 1. Probe lengths are
listed in kilobases (kb). The probe coordinates are
relative coordinates along the region of interest.

| | Begin probe | End probe | Probe length | |
|---|---|---|---|---|
| Probe ID | coordinate | coordinate | (kb) | Color |
| P1 | 0 | 191999 | 192 | Red |
| P2 | 194000 | 231999 | 38 | Cyan |
| P3 | 318000 | 321999 | 4 | Blue |
| P4 | 330000 | 337999 | 8 | Blue |
| P5 | 362000 | 369999 | 8 | Green |
| P6 | 398000 | 401999 | 4 | Red |
| P7 | 402000 | 405999 | 4 | Blue |
| P8 | 406000 | 409999 | 4 | Red |
| P9 | 410000 | 413999 | 4 | Blue |
| P10 | 430000 | 453999 | 24 | Yellow |
| P11 | 494000 | 501999 | 8 | Red |
| P12 | 520000 | 529999 | 10 | Magenta |
| P13 | 534000 | 543999 | 10 | Magenta |
| P14 | 546000 | 553999 | 8 | Magenta |
| P15 | 564000 | 661999 | 98 | Green |

FIG. 13B presents examples of experimental signals obtained by molecular combing and hybridization of the probe pattern for analysis of large rearrangements in the region containing a gene and 5 of its pseudogenes.

The foregoing disclosure provides examples of specific embodiments. As will be understood by those skilled in the art, the approaches, methods, techniques, materials, devices, and so forth disclosed herein may be embodied in additional embodiments as understood by those of skill in the art, it is the intention of this application to encompass and include such variations. Accordingly, this disclosure is illustrative and should not be taken as limiting the scope of the claims. Non-limited embodiments of the invention include:
1. A method for designing color-coded Genetic Morse Code (“GMC”) probe(s)
   (i) comprising:
      (A) identifying a sequence of a nucleic acid target region of interest in a genomic, chromosomal or other nucleic acid sample,
      (B) subdividing the sequence of the target region of interest by defining a set of subsequences,
      (C) identifying duplicate subsequences in the set of defined subsequences inside the target region of interest,
      (D) designing the minimal set of GMC probe(s) that bind to the full nucleic acid target region of interest, wherein said designed GMC probe(s) produce a unique or characteristic color pattern when bound to the nucleic acid target region of interest; and
      (E) synthesizing said designed GMC probe(s); or alternatively
   (ii) comprising:
      (A) identifying a sequence of a nucleic acid target region of interest in a genomic, chromosomal or other nucleic acid sample,
      (B) subdividing the sequence of the target region of interest by defining a set of subsequences,
      (C) identifying duplicate subsequences in the set of defined subsequences inside the target region of interest,
      (D) designing GMC probe(s) that bind to the nucleic acid target region of interest but that do not bind to the duplicate subsequences or that identify duplicate sequences with one or more specific colors, wherein said designed GMC probe(s) produce a unique or characteristic color pattern when bound to the nucleic acid target region of interest; and
      (E) optionally, synthesizing said designed GMC probe(s).
2. The method of embodiment 1, further comprising (F), binding the designed and synthesized probe(s) to a genomic DNA molecule.
3. The method of embodiment 1 or 2, further comprising identifying duplicate subsequences outside the sequence of a target region of interest and (D) designing GMC probe(s) that bind to the nucleic acid target region of interest and adjacent regions but that do not bind to the duplicate subsequences, wherein said designed GMC probe(s) produce a unique or characteristic color pattern when bound to the nucleic acid target region of interest and adjacent regions.
4. The method of any one of embodiments 1, 2 or 3, wherein the GMC probe(s) bind to the nucleic acid target region of interest and to additional nucleic acid region(s) adjacent to duplicate subsequences out of the region of interest, thus forming longer subsequence(s) which can be uniformly coded with a single color so that the designed GMC probe(s) can be distinguished from the smaller defined subdivided subsequences in the target region of interest and from artefactual sequences outside of the sequence of the target region of interest.
5. The method of any one of embodiments 1-4, further comprising identifying interspersed repeats and/or low complexity sequences in the sequence of the nucleic acid target region of interest using RepeatMasker or another bioinformatics database.
6. The method of any one of embodiments 1-5, further comprising identifying segmental duplications in the sequence of the nucleic acid target region of interest using BLAST, BLAT, FASTA, MUSCLE, CLUSTAL or another genome assembly algorithm.
7. The method of any one of embodiments 1 to 6, wherein the nucleic acid molecule is DNA.
8. The method of any one of embodiments 1 to 7, wherein the nucleic acid molecule is genomic DNA.
9. The method of any one of embodiments 1 to 7, wherein the nucleic acid molecule is cDNA.
10. The method of any one of embodiments 1 to 6, wherein the nucleic acid is RNA.
11. The method of any one of embodiments 1 to 10, further comprising sequencing the nucleic acid target region of interest.
12. The method of any one of embodiments 1 to 11, wherein the sequence of the nucleic acid target region of interest is obtained from a sequence database or from a sequence given by a nucleic acid accession number.
13. The method of any one of embodiments 1 to 12, wherein the color-coding of the GMC probe(s) is selected so as to provide a unique color pattern when hybridized to the nucleic acid target region of interest.
14. The method of any one of embodiments 1 to 13, wherein the color-coding of the GMC probe(s) is provided by an algorithm that generates a unique color coding for the target sequence, thus permitting non-ambiguous localization of signals from a locus or loci of interest in the target sequence, whether or not the target nucleic acid is fragmented by DNA breakage during extraction; wherein said unique color coding unambiguously identifies the target sequence from other sequences in the same genomic, chromosomal or other nucleic acid sample.
15. The method of any one of embodiments 1 to 14, wherein the duplicated sequence(s) is at least one selected from the group consisting of terminal repeats, tandem repeats which may be direct repeats, or inverted repeats, satellite DNA, such as that found in centromeres or heterochromatin, minisatellite DNA, for example repeated units of about 10 to 60 base pairs, microsatellite DNA, for example, repeated units of 6-8 or less than 10 base pairs, including those found in telomers, interspersed repeats or interspersed nuclear elements, including DNA transposons (HERVs), retrotransposons, LTR-retrotransposons, non-LTR retrotransposons, including SINEs, LINEs, and SVAs.
16. The method of any one of embodiments 1-15, wherein the target nucleic acid sequence is a subsequence of a chromosomal or genomic DNA, and wherein a set of the color-coded GMC probes further comprises color-coded probes hybridizing to duplicated or non-duplicated sequences outside of said subsequence of the nucleic acid target region of interest.
17. The method of any one of embodiments 1-16, wherein a set of the color-coded GMC probes further comprises probes that recognize duplicated sequences outside the nucleic acid target region of interest that is a region of genomic DNA, and optionally, distinguishing these duplicated sequences from those of the targeted nucleic acid region of interest during a subsequence downstream analysis.
18. The method of any one of embodiments 1 to 17, wherein the target nucleic acid sequence is associated with a genetic disease, disorder or other condition.
19. The method of any one of embodiments 1 to 18, wherein the color-encoded GMC probe(s) uniquely identify a target locus or target loci associated with replication, nucleic acid repair or nucleic acid epigenetics.
20. The method of any one of embodiments 1 to 19, wherein the color-encoded GMC probe(s) uniquely identify a target sequence associated with a genetic disease, disorder or other condition and/or that uniquely identifies a target sequence associated with a normal phenotype.
21. GMC probe(s), in particular color-coded or labelled GMC probe(s), designed by the method according to any one of embodiments 1 to 20.
22. A method for molecular combing comprising contacting a nucleic acid molecule of interest with the GMC probe(s) according to embodiment 21.
23. The method of embodiment 22, wherein the specificity of the GMC probe(s) for the nucleic acid target region of interest sequence is higher than that of a GMC probe that is designed without deleting duplicate subsequences and/or without designing additional probe(s) next to the duplicated subsequences out of the target region which can be uniformly coded with a single color.
24. A method for making a set of Genomic Morse Code (“GMC”) probes that hybridize to non-repeated loci of a nucleic acid target region of interest and produce a unique or characteristic color pattern when hybridized comprising:
   obtaining a sequence of a nucleic acid target region of interest or a portion thereof comprising a target nucleic acid sequence,
   analyzing the sequence and identifying subsequences containing duplicated sequences, and
   producing a set of color-coded GMC probes that do not hybridize to one or more identified duplicated subsequences.
25. The method of embodiment 24, wherein the nucleic acid molecule is DNA.
26. The method of at least one of embodiments 24 or 25, wherein the nucleic acid molecule is genomic DNA.
27. The method of at least one of embodiments 24 or 25, wherein the nucleic acid molecule is cDNA.
28. The method of embodiment 24, wherein the nucleic acid is RNA.
29. The method of at least one of embodiments 24-28, further comprising sequencing the nucleic acid molecule or the portion thereof comprising the target nucleic acid sequence.
30. The method of at least one of embodiments 24-29, wherein the sequence of the nucleic acid molecule or portion comprising the target nucleic acid sequence is obtained from a sequence database or from a sequence given by a nucleic acid accession number.
31. The method of at least one of embodiments 24-30, wherein the target sequence is analyzed using a bioinformatic program.
32. The method of at least one of embodiments 24-31, wherein the sequence of the nucleic acid molecule is analyzed using BLAST, BLAT, FASTA, MUSCLE, CLUSTAL, Tandem Repeats Finder (Benson, Nuc. Acid Res. 27(2):573: 1999) or other similar programs. The most recent prefiling version of each of these programs as well as past versions are known by those skilled in the art and are also hereby incorporated by reference.
33. The method of at least one of embodiments 24-32, wherein the set of color-coded GMC probes is produced using an algorithm that generates a unique color coding for the target sequence, and which does not contain excluded duplicate sequences or subsequences, thus permitting non-ambiguous localization of signals from loci of interest in the target sequence, whether or not the target nucleic acid is fragmented by DNA breakage during extraction; wherein said unique color coding unambiguously identifies the target sequence from other sequences in the same isolated nucleic acid sample.
34. The method of at least one of embodiments 24-33, wherein the set of color-coded GMC probes does not hybridize to at least one duplicate region in the portion of the target nucleic acid molecule corresponding to the target sequence.
35. The method of at least one of embodiments 24-34, wherein the set of color-coded GMC probes does not hybridize to any duplicate regions in the portion of the target nucleic acid molecule corresponding to the target sequence.
36. The method of at least one of embodiments 24-35, wherein at least one member of the set of color-coded GMC probes binds to duplicate sequence(s) in the target sequence.
37. The method of at least one of embodiments 24-36, wherein the duplicated sequence(s) is at least one selected from the group consisting of terminal repeats, tandem repeats which may be direct or inverted repeats, satellite DNA, such as that found in centromeres or heterochromatin, minisatellite DNA, for example repeated units of about 10 to 60 base pairs, microsatellite DNA, for example, repeated units of 6-8 or less than 10 base pairs, including those found in telomers, interspersed repeats or interspersed nuclear elements, including DNA transposons (HERVs), retrotransposons, LTR-retrotransposons, non-LTR retrotransposons, including SINEs, LINEs, and SVAs.
38. The method of at least one of embodiments 24-37, wherein the target nucleic acid sequence is a subsequence of a chromosomal or genomic DNA, and wherein the set of color-coded GMC probes further comprises color-coded probes hybridizing to repeated or non-repeated sequences outside of said subsequence of chromosomal or genomic DNA.
39. The method of at least one of embodiments 24-38, wherein the set of color-coded GMC probes further comprises probes that recognize duplicated sequences outside a targeted genomic region and, optionally, distinguishing these duplicated sequences from those of the targeted genomic regions during a subsequence downstream analysis.
40. The method of at least one of embodiments 24-39, wherein the target nucleic acid sequence is associated with a genetic disease, disorder or other condition.
41. The method of at least one of embodiments 24-40, wherein the set of color-encoded probes uniquely identifies a target locus or target loci associated with replication, nucleic acid repair or nucleic acid epigenetics.
42. The method of at least one of embodiments 24-41, wherein the set of color-encoded probes uniquely identifies a target sequence associated with a genetic disease, disorder or other condition and/or that uniquely identifies a target sequence associated with a normal phenotype.
43. The method of at least one of embodiments 24-42, further comprising contacting a target nucleic acid molecule with the set of color-coded GMC probes.
44. The method of at least one of embodiments 24-34, further comprising performing molecular combing using the set of color-coded probes.
45. GMC probe(s), in particular color-coded or labelled GMC probe(s), designed by the method according to any one of embodiments 24-44.
46. A method for designing a color-coded GMC probe(s) comprising:
   (A) identifying a sequence of a nucleic acid target region of interest in a genomic, chromosomal or other nucleic acid sample,
   (B) subdividing the sequence of the target region of interest by defining a set of subsequences,
   (C) identifying duplicate subsequences in the set of defined subsequences,
   (D) identifying duplicate subsequences outside the sequence of a target region of interest,
   (E) GMC probe(s) that either bind to the nucleic acid target region of interest where duplicate subsequences were deleted or that bind both to the nucleic acid target region of interest and to additional nucleic acid region(s) adjacent to duplicate subsequences out of the region of interest, thus forming longer subsequence(s) which can be uniformly coded with a single color so that the designed GMC probe(s) can be distinguished from the smaller defined subdivided subsequences in the target region of interest and from artefactual sequences outside of the sequence of the target region of interest
   (F) selecting a unique pattern of color encoding for the GMC probe(s), and optionally synthesizing or otherwise producing the GMC probes.
47. The method of embodiment 46, wherein RepeatMasker database or another bioinformatics database is used to identify interspersed repeats and low complexity sequences in the nucleic acid target region of interest.
48. The method of any one of embodiments 46 or 47, wherein BLAST, BLAT, FASTA, MUSCLE, CLUSTAL or another genome assembly algorithm is used to identify segmental duplications in the nucleic acid target region of interest. In case of ambiguity in describing algorithms, computer programs, databases, or accession numbers which may be updated over time, the last available version nearest the filing date of this disclosure should be used.
49. GMC probe(s), in particular color-coded or labelled GMC probe(s), designed or produced by the method according to any one of embodiments 46-48.
50. A method for molecular combing comprising contacting a nucleic acid molecule of interest with the GMC probe(s) according to any one embodiments 21, 45, 49, 52 or 59.
51. A method for removing problematic nucleic acid subsequences, which can be misinterpreted as informative about the region of interest containing them; comprising:
   (A) analyzing a sequence of a region of interest by defining a set of smaller fragments whose lengths are defined by a length parameter that avoids sequences rich in repeat elements using an online data base such as RepeatMasker;
   (B) identifying segmental duplications in said set of smaller fragments using BLAST, BLAT, FASTA, MUSCLE, CLUSTAL or another genome assembly or multiple sequence alignment algorithm;
   (C) removing from the set of fragments, fragment(s) that can be completely covered by duplications from other fragments, and, optionally, identifying color-constraints for color-encoding the remaining fragments;
   (D) merging separated duplications identified by the genome assembly algorithm when the duplications are distanced by less than a proportion of the combination of their lengths, and filtering the duplications by homology and length based on selected parameter values;
   (E) whose lengths are selected to minimize the number of fragments containing fragments containing repeat subsequences using RepeatMasker or another bioinformatic program;
   (F) the regions into smaller fragments of size specified by a parameter. Depending on the labelling technique applied, either genetic fragments of several kilobases, or oligonucleotide fragments of dozens of basepairs can be defined. If specified, this step optimizes fragment definitions to avoid sequences rich in repeat elements from design using online data bases such as RepeatMasker [Jurka, J, 2000; Smit A F A, 1996-2010]. The constraints of feasibility for synthesis or amplification of the resulting fragments are not considered.
52. Color-coded or labelled GMC probe(s) that exclude polynucleotide sequences that are part of segmental duplications and/or generate patterns, when bound to a region of interest in a target DNA sequence, that enable discrimination between the region of interest and duplicated loci on the target DNA sequence; wherein specificity of the color-coded GMC probe(s) for the target nucleic acid sequence is higher than that of a GMC probe(s) that is designed without deleting duplicate subsequences and/or without the design of an additional probe adjacent to duplicate subsequences out of the region of interest which can be uniformly coded with a single color.
53. A target nucleic acid to which the color-coded or labelled GMC probe(s) of embodiment 21, 45, 49, 52 or 59 have been bound and which exhibits a characteristic or unique color or labelling pattern.
54. Use of the color-coded or labelled GMC probe(s) according to embodiments 21, 45, 49, or 52 for detection of a target locus or target loci associated with replication, nucleic acid repair or nucleic acid epigenetics or for detection of a target sequence associated with a genetic disease, disorder or other condition and/or that uniquely identifies a target sequence associated with a normal phenotype, and optionally for diagnosis of a disease, disorder of condition associated with a particular arrangement or rearrangement of genomic DNA.
55. A method for producing a pattern of color-coded probes comprising the steps:
   (A) identifying a sequence of a nucleic acid target region of interest in a genomic, chromosomal or other nucleic acid sample,
   (B) subdividing the sequence of the target region of interest by defining a set of subsequences,
   (C) identifying duplicate subsequences in the set of defined subsequences,
   (D) identifying duplicate subsequences outside the sequence of a target region of interest,
   (E) designing GMC probe(s) that either bind to the nucleic acid target region of interest and produce a characteristic or unique color pattern, but deletes duplicate subsequences and/or that bind both to the nucleic acid target region of interest and to additional nucleic acid region(s) adjacent to duplicate subsequences out of the region of interest, thus forming longer subsequence(s) which can be uniformly coded with a single color so that the designed GMC probe(s) can be distinguished from the smaller defined subdivided subsequences in the target region of interest and from artefactual sequences outside of the sequence of the target region of interest,
   (F) selecting a unique pattern of color encoding for the GMC probe(s), and
   (G) contacting said color-encoded GMC probe(s) with the target nucleic acid region of interest thereby painting the target nucleic acid region of interest with a characteristic or unique color pattern.
   (H) Analyzing the hybridization product obtained in step (G)
56. The method of embodiment 55, where unicity of partial color patterns is guaranteed over all regions
57. The method of embodiment 55, where said steps A to H also consider cross-duplications among multiple target regions for definition of GMC probe(s).
58. The method of any one of embodiments 1-20, 22-44, 46-48, 50, 51, 54-57, wherein the method is applied simultaneously on multiple target regions or on multiple nucleic acid sequences.
59. Color-coded or labelled GMC probe(s) which were designed in order to insure unicity of partial sequences of GMC probe(s) containing subparts of color-coded probe(s), when bound to a region of interest in a target DNA or nucleic acid sequence, that enable unambiguous loci localization of partial GMC sequences along the GMC probe(s); wherein specificity of the partial nucleotidic sequences of color-coded GMC probe(s) for the target nucleic acid sequence is higher than that of a GMC probe(s) that is designed without analysis and constraint on the unicity of such partial sequences.
60. Use of the color-coded or labelled GMC probe(s) according to embodiments 21, 45, 49, 52 and/or 59 for detection of expected and off-target rearrangements or genetic modifications produced by gene editing methods.
61. A kit for the detection of at least one domain or loci of interest in genomic DNA or other target nucleic acid containing color-coded or labelled GMC probe(s) according embodiments 21, 45, 49, 52 or 59 and optionally, equipment and reagents for sample preparation such as DNA extraction equipment that provides purified, very high molecular weight DNA (e.g., median size of 100 kb) suitable for Molecular Combing; equipment and reagents for Molecular Combing, such as a vinyl silane treated glass surface (e.g., a coverslip) and equipment or a system for stretching DNA; equipment and devices (e.g., a scanner) for reading target DNA contacted with GMC probe(s), software or computer equipment for analyzing, processing and storing these data, packaging materials, and/or instructions for use.
62. Use of the color-coded or labelled GMC probe(s) according to embodiments 21, 45, 49, 52 or 59 for detection of the locus for Spinal Muscular Atrophy (SMA locus).
63. Use of the color-coded or labelled GMC probe(s) according to embodiments 21, 45, 49, 52 or 59 for detection of large rearrangements in nucleic acid of the region involved in the hereditary nonpolyposis colon cancer (HNPCC), in particular in the region encompassing 2 genes involved in HNPCC, MLH1 and PMS2.

### Terminology

Terminology used herein is for the purpose of describing particular embodiments only and is not intended to be limiting of the invention.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

Links are disabled by deletion of http: or by insertion of a space or underlined space before www. In some instances, the text available via the link on the “last accessed” date may be incorporated by reference.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all subranges or intermediate values subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Although the terms “first” and “second” may be used herein to describe various features/elements (including steps), these features/elements should not be limited by these tennis, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

### BIBLIOGRAPHY

Lebofsky, Ronald, et al. “DNA replication origin interference increases the spacing between initiation events in human cells.” Molecular biology of the cell 17.12 (2006): 5337-5345.
Gad, Sophie, et al. “Color bar coding the BRCA1 gene on combed DNA: a useful strategy for detecting large gene rearrangements.” Genes, Chromosomes and Cancer 31.1 (2001): 75-84.
Gad, Sea, et al. “Bar code screening on combed DNA for large rearrangements of the BRCA1 and BRCA2 genes in French breast cancer families.” Journal of medical genetics 39.11 (2002): 817-821.
Puget, Nadine, et al. “Distinct BRCA1 rearrangements involving the BRCA1 pseudogene suggest the existence of a recombination hot spot.” The American Journal of Human Genetics 70.4 (2002): 858-865.
Gad, Sophie, et al. “Identification of a large rearrangement of the BRCA1 gene using color bar code on combed DNA in an American breast/ovarian cancer family previously studied by direct sequencing.” Journal of medical genetics 38.6 (2001): 388-392.
Cheeseman, Kevin, et al. “A diagnostic genetic test for the physical mapping of germline rearrangements in the susceptibility breast cancer genes BRCA1 and BRCA2.” Human mutation 33.6 (2012): 998-1009.
Michalet, Xavier, et al. “Dynamic molecular combing: stretching the whole human genome for high-resolution studies.” Science 277.5331 (1997): 1518-1523.
Herrick, John, et al. “Quantifying single gene copy number by measuring fluorescent probe lengths on combed genomic DNA.” Proceedings of the National Academy of Sciences 97.1 (2000): 222-227.
Beliveau, Brian J., et al. “Versatile design and synthesis platform for visualizing genomes with Oligopaint FISH probes.” Proceedings of the National Academy of Sciences 109.52 (2012): 21301-21306.
Bienko, Magda, et al. “A versatile genome-scale PCR-based pipeline for high-definition DNA FISH.” Nature methods 10.2 (2013): 122-124.
Komatsu, Jun et al. (2016) Method for identifying or detecting genomic rearrangements in a biological sample. U.S. Pat. No. 9,133,514 B2. Genomic Vision (Bagneux, FR).
Lebofsky, Ronald et al. (2007) Genomic Morse code. U.S. Pat. No. 7,985,542 B2: Institut Pasteur (Paris, FR).
Jing, Junping, et al. “Automated high resolution optical mapping using arrayed, fluid-fixed DNA molecules” Proceedings of the National Academy of Sciences 95 (1998): 8046-51
Swennenhuis, Joost F., et al. “Construction of repeat-free fluorescence in situ hybridization probes.” Nucleic acids research 40.3 (2012): e20-e20.
Gal, Joseph G and Pardue, Marie Lou. “Formation and detection of RNA-DNA hybrid molecules in cytological preparations” Proceedings of the National Academy of Sciences 63.2 (1969): 378-83.
Bauman, J. G. J. et al. “A new method for fluorescence microscopical localization of specific DNA sequences by in situ hybridization of fluorochrome-labelled RNA” Experimental Cell Research 128.2 (1980): 485-90.
McCaffrey, Jennifer, et al. “CRISPR-CAS9 D10A nickase target-specific fluorescent labeling of double strand DNA for whole genome mapping and structural variation analysis.” Nucleic acids research (2015): gkv878.
Flicek, Paul, and Ewan Birney. “Sense from sequence reads: methods for alignment and assembly.” Nature methods 6 (2009): S6-S12.
Hastie, Alex R., et al. “Rapid genome mapping in nanochannel arrays for highly complete and accurate de novo sequence assembly of the complex Aegilops tauschii genome.” PloS one 8.2 (2013): e55864.
Pearson W R, Lipman D J. Improved tools for biological sequence comparison. Proc Natl Acad Sci USA. 1988 April; 85(8):2444-8.
Kent W J. BLAT—the BLAST-like alignment tool. Genome Res. 2002 April; 12(4):656-64
Jurka J. Repbase update: a database and an electronic journal of repetitive elements. Trends Genet. 2000 September; 16(9):418-420. PMID: 10973072
Smit A F A, Hubley R, Green P. RepeatMasker Open-3.0. http://_www.repeatmasker.org. 1996-2010.
Edgar, Robert C. “MUSCLE: multiple sequence alignment with high accuracy and high throughput.” Nucleic acids research 32.5 (2004): 1792-1797.
Chenna, Ramu, et al. “Multiple sequence alignment with the Clustal series of programs.” Nucleic acids research 31.13 (2003): 3497-3500.
Benson, Gary. “Tandem repeats finder: a program to analyze DNA sequences.” Nucleic acids research 27.2 (1999): 573.
Warburton, Peter E., et al. “Inverted repeat structure of the human genome: the X-chromosome contains a preponderance of large, highly homologous inverted repeats that contain testes genes.” Genome research 14.10a (2004): 1861-1869.
Komatsu, Jun et al. (2016) Method for identifying or detecting genomic rearrangements in a biological sample. U.S. Pat. No. 9,133,514 B2. Genomic Vision (Bagneux, FR).
Lebofsky, Ronald et al. (2007) Genomic morse code. U.S. Pat. No. 7,985,542 B2: Institut Pasteur (Paris, FR)
Pierret M., et al. Molecular combing reveals structural variations in the Spinal Muscular Atrophy locus in African-American population; (Abstract/Program 850 W). Presented at the 66th Annual Meeting of The American Society of Human Genetics, Date, Location (e.g., Oct. 19, 2016, Vancouver, Canada).
Rosenbloom K R, et al. « The UCSC Genome Browser database: 2015 update. » Nucleic Acids Res. (2015) January
GV (2016) http://_www.genomicvision.com/products/genetic-tests/hnpcc/ (last accessed Nov. 28, 2016)
Genome Browser (2017) described by and incorporated by reference to text available at https://_genome.ucsc.edu/ (last accessed Nov. 23, 2017).

1. A method for designing color-coded Genetic Morse Code (“GMC”) probe(s) comprising:
(A) identifying a sequence of a nucleic acid target region of interest in a genomic, chromosomal or other nucleic acid sample,
(B) subdividing the sequence of the target region of interest by defining a set of subsequences,
(C) identifying duplicate subsequences in the set of defined subsequences inside the target region of interest,
(D) designing the minimal set of GMC probe(s) that bind to the full nucleic acid target region of interest, wherein said designed GMC probe(s) produce a unique or characteristic color pattern when bound to the nucleic acid target region of interest; and
(E) synthesizing said designed GMC probe(s).

2. The method according to claim 1, further comprising (F), binding the designed and synthesized probe(s) to a genomic DNA molecule.

3. The method according to claim 1, further comprising identifying duplicate subsequences outside the sequence of a target region of interest and (D) designing GMC probe(s) that bind to the nucleic acid target region of interest and adjacent regions but that do not bind to the duplicate subsequences or that identify duplicate sequences with one or more specific colors, wherein said designed GMC probe(s) produce a unique or characteristic color pattern when bound to the nucleic acid target region of interest and adjacent regions.

4. The method according to claim 1, further comprising identifying interspersed repeats and/or low complexity sequences in the sequence of the nucleic acid target region of interest using RepeatMasker or another bioinformatics database.

5. The method according to claim 1, further comprising identifying segmental duplications in the sequence of the nucleic acid target region of interest using BLAST, BLAT, FASTA, MUSCLE, CLUSTAL or another genome assembly algorithm.

6. The method according to claim 1, wherein the color-coding of the GMC probes is selected so as to provide a unique color pattern when hybridized to the nucleic acid target region of interest.

7. The method according to claim 1, wherein the color-coding of the GMC probe(s) is provided by an algorithm that generates a unique color coding for the target sequence, thus permitting non-ambiguous localization of signals from a locus or loci of interest in the target sequence, whether or not the target nucleic acid is fragmented by DNA breakage during extraction; wherein said unique color coding unambiguously identifies the target sequence from other sequences in the same genomic, chromosomal or other nucleic acid sample.

8. The method according to claim 1, wherein the duplicate subsequence(s) are at least one selected from the group consisting of terminal repeats, tandem repeats, direct repeats, inverted repeats, satellite DNA, minisatellite DNA, microsatellite DNA, interspersed repeats or interspersed nuclear elements, DNA transposons (HERVs), retrotransposons, LTR-retrotransposons, non-LTR retrotransposons, SINEs, LINEs, and SVAs.

9. The method according to claim 1, wherein the target nucleic acid sequence is a subsequence of a chromosomal or genomic DNA, and wherein a set of the color-coded GMC probes further comprises color-coded probes hybridizing to duplicated or non-duplicated sequences outside of said subsequence of the nucleic acid target region of interest.

10. The method according to claim 1, wherein a set of the color-coded GMC probes further comprises probes that recognize duplicated sequences outside the nucleic acid target region of interest that is a region of genomic DNA, and optionally, distinguishing these duplicated sequences from those of the targeted nucleic acid region of interest during a subsequence downstream analysis.

11. The method according to claim 1, wherein the target nucleic acid sequence is associated with a genetic disease, disorder or other condition.

12. The method according to claim 1, wherein the color-encoded GMC probe(s) uniquely identify a target locus or target loci associated with replication, nucleic acid repair or nucleic acid epigenetics.

13. The method according to claim 1, wherein the color-encoded GMC probe(s) uniquely identify a target sequence associated with a genetic disease, disorder or other condition and/or that uniquely identifies a target sequence associated with a normal phenotype.

14. Color-coded or labelled GMC probe(s) designed or produced by the method according to claim 1.

15. A method for molecular combing comprising contacting a nucleic acid molecule of interest with the GMC probe(s) according to claim 14.

16. The method according to claim 15, wherein the specificity of the GMC probe(s) for the nucleic acid target region of interest sequence is higher than that of a GMC probe that is designed without deleting duplicate subsequences and/or without designing additional probe(s) next to the duplicated subsequences out of the target region which can be uniformly coded with a single color.

17. A method for producing a pattern of color-coded probes comprising the steps:
(A) identifying a sequence of a nucleic acid target region of interest in a genomic, chromosomal or other nucleic acid sample,
(B) subdividing the sequence of the target region of interest by defining a set of subsequences,
(C) identifying duplicate subsequences in the set of defined subsequences,
(D) identifying duplicate subsequences outside the sequence of a target region of interest,
(E) designing GMC probe(s) that either bind to the nucleic acid target region of interest and produce a characteristic or unique color pattern, but deletes duplicate subsequences and/or that bind both to the nucleic acid target region of interest and to additional nucleic acid region(s) adjacent to duplicate subsequences out of the region of interest, thus forming longer subsequence(s) which can be uniformly coded with a single color so that the designed GMC probe(s) can be distinguished from the smaller defined subdivided subsequences in the target region of interest and from artefactual sequences outside of the sequence of the target region of interest,
(F) selecting a unique pattern of color encoding for the GMC probe(s), and
(G) contacting said color-encoded GMC probe(s) with the target nucleic acid region of interest thereby painting the target nucleic acid region of interest with a characteristic or unique color pattern.
(H) analyzing the hybridization product obtained in step (G)

18. The method according to claim 17, where unicity of partial color patterns is guaranteed over all regions.

19. The method according to claim 17, where said steps A to H also consider cross-duplications among multiple target regions for definition of GMC probe(s).

20. The method according to claim 17, wherein the method is applied simultaneously on multiple target regions or on multiple nucleic acid sequences.
